(19) United States
(12) Patent Application Publication
(10) Pub. No.: US 2005/0221314 A1
(43) Pub. Date: Oct. 6, 2005

(54) Method and device for determination of tissue specificity of free floating dna in bodily fluids

(75) Inventors: Kurt Berlin, Stahnsdorf (DE); Andrzej Sledziewski, Shoreline, WA (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Epigenomics AG, Berlin (DE)

(21) Appl. No.: 10/506,693

(22) PCT Filed: Mar. 5, 2003
(86) PCT No.: PCT/EP03/02245

§ 371 Date: Apr. 21, 2005

(30) Foreign Application Priority Data
Mar. 5, 2002 (EP) 02004954.0

(51) Int. Cl. C12Q 1/68

(52) U.S. Cl. 435/6

(57) ABSTRACT

The present invention relates to methods for detecting free floating nucleic acids, as present in not cellular bound nucleic acids in bodily fluids like plasma or serum fractions of human or animal blood or in any other tissue samples derived from the human or animal body in order to diagnose a cell proliferative disease. Specifically the invention relates to the detection of increased levels of nucleic acids in bodily fluids. Furthermore the invention allows to determine the source of the enriched DNA by measuring the ratio of DNA originating from a certain organ versus total DNA from other organs in a given bodily fluid sample by specifying the DNA's methylation pattern. This can be done with or without increasing the DNA concentration of a given biological sample. In a preferred embodiment a further analysis of this methylation pattern allows for the detection of the presence of tumourous or otherwise proliferative disease in said organ.

### BACKGROUND OF THE INVENTION

This invention relates to methods for detecting free floating nucleic acids, as present in not cellular bound nucleic acids in bodily fluids like plasma or serum fractions of human or animal blood or in any other tissue samples derived from the human or animal body in order to diagnose a cell proliferative disease. Specifically the invention relates to the detection of increased levels of nucleic acids in bodily fluids. Furthermore the invention allows to determine the source of the enriched DNA by measuring the ratio of DNA originating from a certain organ versus total DNA from other organs in a given bodily fluid sample by specifying the DNA's methylation pattern. This can be done with or without increasing the DNA concentration of a given biological sample. In a preferred embodiment a further analysis of this methylation pattern allows for the detection of the presence of tumourous or otherwise proliferative disease in said organ.

### PRIOR ART

DNA Based Assays to Detect Cancer

A number of genetic alterations like mutations in certain genes, but also loss of heterozygosity and microsatellite instability at certain loci can be detected in DNA samples from tumour tissue. These DNA alterations can be detected in DNA retrieved from the tumour tissue of a patient. In some cases it has been reported that these alterations were also found in DNA samples from serum or blood or the sputum of those tumour patients.

It is known that cigarette smokers have increased bronchial secretions that contain exfoliated cells from the bronchial tree. From analysing these excreted cells, premalignant cytological changes could be detected several years before a clinical diagnosis of lung cancer in high risk patients (Saccomanno et al. (1974) Cancer (Phila.), 33: 256-270). These studies were not easily reproducible and required specific skills in the person which analysed those samples. Therefore, to enhance the predictive value of the sputum samples, it has been suggested to use molecular assays, for example to detect mutations within the K-ras gene or microsatellite alterations specific to the tumour (Mao et al. (1994) Proc. Natl. Acad. Sci. USA, 91: 9871-9875 and Mao et al. (1994) Cancer Res., 54: 1634-1637). K-ras as well as p53 mutations have been detected in bodily fluids as found in cytological samples of the sputum and bronchial lavage of lung cancer patients and chronic smokers (Kersting et al. (2000) J. Clin. Oncol., 18: 3221-3229 and Ahrendt et al. (1999) J. Natl. Cancer Inst. (Bethesda), 91: 332-339). Knowing the nucleic acid sequences of specific marker genes involved in a certain type of cancer like, for example, lung cancer enabled the analysis of these sputum samples and allowed to predict the development of lung cancer in high risk patients. More relevant information on this matter can be found in patent WO 95/16792 by Maurice Stroun, Philippe Anker and Valeri Vasioukhin. However, these methods are not ideal as they lack sensitivity and the overall prevalence of these changes in non-small cell lung cancer is less than 25% (Palmisano et al. (2000), Cancer Res. 60: 5954-5958). Also for prostate cancer it has been reported that the inactivation of the HPC2/ELAC2 gene via LOH is a relatively uncommon event (Wu et al. (2001) Cancer Res 61: 8651-8653). Another factor highly correlated with the occurrence of tumors is the hypermethylation of certain promoters and promoter regions.

Methylation

In recent decades in molecular biology studies have focussed primarily on genes, the translation of those genes into RNA, and the transcription of the RNA into protein. There has been a more limited analysis of the regulatory mechanisms associated with gene control. Gene regulation, for example, at what stage of development of the individual a gene is activated or inhibited, and the tissue specific nature of this regulation is less understood. However, it can be correlated with a high degree of probability to the extent and nature of methylation of the gene or genome. From this observation it is reasonable to infer that pathogenic genetic disorders may be detected from irregular genetic methylation patterns and this has been shown for a number of cases. In addition this invention discloses a method on how to determine the origin of DNA in a bodily fluid by analyzing its methylation pattern in order to detect aberrant levels of DNA deriving from a certain organ, indicating a cell proliferative disease of said organ.

In higher order eukaryotes DNA is methylated nearly exclusively at cytosines located 5′ to guanosine in the CpG dinucleotide. This modification has important regulatory effects on gene expression, especially when involving CpG rich areas, known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosome of females. Aberrant methylation of normally unmethylated CpG islands has been described as a frequent event in immortalised and transformed cells, and has been associated with transcriptional inactivation of defined tumour suppressor genes in human cancers.

Human cancer cells typically contain somatically altered genomes, characterised by mutation, amplification, or deletion of critical genes. In addition, the DNA template from human cancer cells often displays somatic changes in DNA methylation (E. R. Fearon, et al., Cell, 61:759, 1990; P. A. Jones, et al., Cancer Res., 46: 461, 1986; R. Holliday, Science, 238: 163, 1987; A. De Bustros, et al., Proc. Natl. Acad. Sci., USA, 85: 5693, 1988; P. A. Jones, et al., Adv. Cancer Res., 54:1, 1990; S. B. Baylin, et al., Cancer Cells, 3 :383, 1991; M. Makos, et al., Proc. Natl. Acad. Sci., USA, 89: 1929, 1992; N. Ohtani-Fujita, et al., Oncogene, 8:1063, 1993). However, the precise role of abnormal DNA methylation in human tumourigenesis has not been established. DNA methylases transfer methyl groups from the universal methyl donor S-adenosyl methionine to specific sites on the DNA.

Several biological functions have been attributed to the methylated bases in DNA. The most established biological function is the protection of the DNA from digestion by cognate restriction enzymes. The restriction modification phenomenon has, so far, been observed only in bacteria. Mammalian cells, however, possess a different methylase that exclusively methylates cytosine residues on the DNA, that are 5′ neighbours of guanine (CpG). This methylation has been shown by several lines of evidence to play a role in gene activity, cell differentiation, tumourigenesis, X-chromosome inactivation, genomic imprinting and other major biological processes (Razin, A., H., and Riggs, R. D. eds. in DNA Methylation Biochemistry and Biological Significance, Springer-Verlag, N.Y., 1984).

Although the exact mechanisms by which DNA methylation effects DNA transcription are unknown, the relationship between disease and methylation has been well documented. Misregulation of genes may be predicted by comparing their methylation pattern with phenotypically ‘normal’ expression patterns. The following are cases of disease associated with modified methylation patterns, the specific role of methylation in cancer is described in the next paragraph:
Hodgkin's disease (Garcia J F et al “Loss of p16 protein expression associated with methylation of the p16INK4A gene is a frequent finding in Hodgkin's disease” Lab invest 1999 December; 79 (12):1453-9)
Prader-Willi/Angelman's syndrome (Zeschnigh et al “Imprinted segments in the human genome: different DNA methylation patterns in the Prader Willi/Angelman syndrome region as determined by the genomic sequencing method” Human Mol. Genetics (1997) (6) 3 pp 387-395)
ICF syndrome (Tuck-Muller et al “CMDNA hypomethylation and unusual chromosome instability in cell lines from ICF syndrome patients” Cytogenet Call Genet 2000; 89 (1-2):121-8
Dermatofibroma (Chen T C et al “Dermatofibroma is a clonal proliferative disease” J Cutan Pathol 2000 January;27 (1):36-9)
Hypertension (Lee S D et al. “Monoclonal endothelial cell proliferation is present in primary but not secondary pulmonary hypertension” J clin Invest 1998 Mar. 1, 101 (5):927-34)
Autism (Klauck S M et al. “Molecular genetic analysis of the FMR-1 gene in a large collection of autistic patients” Human Genet 1997 August; 100 (2): 224-9)
Fragile X Syndrome (Hornstra I K et al. “High resolution methylation analysis of the FMR1 gene trinucleotide repeat region in fragile X syndrome” Hum Mol Genet 1993 October, 2 (10):1659-65)
Huntigton's disease (Ferluga J et al. “possible organ and age related epigenetic factors in Huntington's disease and colorectal carcinoma” Med hypotheses 1989 May;29 (1);51-4

All of documents cited herein are hereby incorporated by reference in their entirety.

Hypermethylation and Cancer

DNA methylation can down-regulate gene expression, and when it does so inappropriately it might lead to a shutting off of tumour suppressor genes, for instance and cause cancer. Consequently, it has been shown frequently that certain regions of the genome are hypermethylated in tumour tissue when this is not the case in neighbouring unaffected cells. A well investigated system is the inactivation of GSTP1 (glutathione-S-transferase promoter 1) by CpG island hypermethylation, the most common somatic genome alteration yet reported for human prostate cancer, occurs early during human prostatic carcinogenesis and results in a loss of GSTP1 caretaker function, leaving prostate cells with inadequate defences against oxidant and electrophile carcinogens. The genetic diagnosis of prostate cancer via detection of the methylation status of the GSTP1 has been described in the patent U.S. Pat. No. 5,552,277. Another example out of many more is described in the following paper: Yanagisawa Y et al. (2000) “Methylation of the hMLH1 promoter in familial gastric cancer with microsatellite instability” Int J Cancer 85:50-3).

A recent example for the correlation between hypermethylation and cancer is given by Maruyama et al. when they reported a positive correlation between the median methylation index of a number of selected genes and the prognosis of bladder cancer development in December 2001 (Maruyama et al. (2001) Cancer Res 61: 8659-8663). Methylation of CDH1, FHIT, and a high MI were associated with shortened survival. CDH1 methylation positive status was independently associated with poor survival in multivariate analyses. The authors conclude that the methylation profile may be a potential new biomarker of risk prediction in bladder cancer, but as they only analysed biopsy samples this would require surgical operation on a patient. However more recent studies have highlighted the possibility to detect DNA methylation in DNA from bodily fluids rather than from tumour tissue itself.

DNA Methylation in Bodily Fluids

For example in DNA from exfoliated cells in sputum samples from lung cancer patients or high risk patients the p16 tumour suppressor gene promoter and/or 06-methylguanine-DNA methyltransferase promoters could be shown to be aberrantly methylated. The aberrant methylation could be detected in DNA from sputum in 100% of patients with squamous cell lung carcinoma up to 3 years before clinical diagnosis (Palmisano et al. (2000), Cancer Res. 60: 5954-5958).

When the methylation status of the p15 and p16 promoter region from tumour DNA and blood (plasma, serum and buffy coat samples) DNA from hepatocellular carcinoma patients was investigated, 87% of the patients with tumour methylation also showed methylated DNA in the blood stream. None of the control samples were methylation positive (Wong et al. (2000) Clin Cancer Res 6 (9):3516-3521). In addition a study on Head and Neck cancer revealed a correlation between serum DNA methylation and tumour DNA methylation of 42% (Sanchez-Cespedes et al. (2000) Cancer Res 60: 892-895).

Methylated DNA as a tumour marker is not only restricted to the sputum or blood stream, but can—at least in prostate carcinoma patients—also be found in urine or ejaculate samples. In this study 94% of the tumour DNA samples were methylated, 72% of the plasma or serum samples, 50% of ejaculate samples and 36% of urine samples (after prostatic massage in order to release prostatic secretions) from patients with prostate cancer whereas no methylation was detected in samples from the control group (Cairns et al. (2001) Clin Cancer Res 7: 2727-2730).

The detection of aberrant promoter region methylation constitutes a promising approach for using DNA methylation based marker assays for the early detection of common human cancers. As hypermethylation is involved in a wide range of cancer types one can think of a number of similar approaches for other types of cancer. For an overview see:
Esteller, M., Corn, P. G., Baylin, S. B., Herman, J. G. (2001). A Gene Hypermethylation Profile of Human Cancer. Cancer Res 61: 3225-3229 or for a selection of recent publications on the matter:
Byun, D.-S., Lee, M.-G., Chae, K.-S., Ryu, B.-G., Chi, S.-G. (2001). Frequent Epigenetic Inactivation of RASSF1A by Aberrant Promoter Hypermethylation in Human Gastric Adenocarcinoma. Cancer Res 61: 7034-7038.
Agathanggelou A., Honorio S., Macartney D. P., Martinez A., Dallol A., Rader J., Fullwood P., Chauhan A., Walker R., Shaw J. A., Hosoe S., Lerman M. I., Minna J. D., Maher E. R., Latif F. (2001). Methylation associated inactivation of RASSF1A from region 3 p21.3 in lung, breast and ovarian tumours. Oncogene, 20: 1509-1518.
Dong, S. M., Kim, H.-S., Rha, S.-H., Sidransky, D. (2001). Promoter Hypermethylation of Multiple Genes in Carcinoma of the Uterine Cervix. Clin Cancer Res 7: 1982-1986.
Herman J. G., Latif F., Weng Y. K., Lerman M. I., Zbar B., Liu S., et al (1994) Silencing of the VHL tumor suppressor gene by DNA methylation in renal carcinomas. Proc. Natl. Acad. Sci. USA, 91: 9700-9704.
   Methods to Detect Methylated DNA

In the previous paragraphs the significance of methylation of certain cytosine bases for gene activity, cell differentiation, tumorigenesis, X-chromosome inactivation, genomic imprinting and other major biological processes (Razin, A., H., and Riggs, R. D. eds. in DNA Methylation Biochemistry and Biological Significance, Springer-Verlag, N.Y., 1984) has been described. The cytosine's modification in form of methylation contains significant information. It is obvious that the identification of 5-methylcytosine in a DNA sequence as opposed to unmethylated cytosine is of greatest importance to analyse its role further. But, because the 5-Methylcytosine behaves just as a cytosine for what concerns its hybridisation preference (a property relied on for sequence analysis) its positions can not be identified by a normal sequencing reaction.

Furthermore, in a PCR amplification this relevant epigenetic information, methylated cytosine or unmethylated cytosine, will be lost completely.

Several methods are known that solve this problem. Usually the genomic DNA is treated with a chemical or enzyme leading to a conversion of the cytosine bases, which consequently allows to differentiate the bases afterwards. Some restriction enzymes are capable of differentiating between methylated and unmethylated DNA.

A relatively new and currently the most frequently used method for analysing DNA for 5-methylcytosine is based upon the specific reaction of bisulfite with cytosine which, upon subsequent alkaline hydrolysis, is converted to uracil, whereas 5-methylcytosine remains unmodified under these conditions (Shapiro et al. (1970) Nature 227: 1047). Uracil corresponds to thymine in its base pairing behaviour, whereas 5-methylcytosine doesn't change its chemical properties under this treatment and corresponds to guanine. Consequently, the original DNA is converted in such a manner that methyl-cytosine, which originally could not be distinguished from cytosine by its hybridisation behaviour, can now be detected as the only remaining cytosine using “normal” molecular biological techniques, for example, by amplification and hybridisation or sequencing. All of these techniques are based on base pairing which can now be fully exploited. Comparing the sequences of the DNA with and without bisulfite treatment allows an easy identification of those bases that have been methylated.

An overview of the further known methods of detecting 5-methylcytosine may be gathered from the following review article: Rein, T., DePamphilis, M. L., Zorbas, H., Nucleic Acids Res. 1998, 26, 2255.

In terms of sensitivity, the prior art is defined by a method, which encloses the DNA to be analysed in an agarose matrix, thus preventing the diffusion and renaturation of the DNA (bisulfite reacts with single-stranded DNA only), and which replaces all precipitation and purification steps with fast dialysis (Olek A, Oswald J, Walter J. (1996) A modified and improved method for bisulphite based cytosine methylation analysis. Nucleic Acids Res. 24: 5064-6). Using this method, it is possible to analyse individual cells, which illustrates the potential of the method.

To date, barring few exceptions (e.g., Zeschnigk M, Lich C, Buiting K, Doerfler W, Horsthemke B (1997) A single-tube PCR test for the diagnosis of Angelman and Prader-Willi syndrome based on allelic methylation differences at the SNRPN locus. Eur J Hum Genet. 5: 94-8) the bisulfite technique is only used in research. Always, however, short, specific fragments of a known gene are amplified subsequent to a bisulfite treatment and either completely sequenced (Olek A, Walter J. (1997) The pre-implantation ontogeny of the H19 methylation imprint. Nat Genet. 3: 275-6) or individual cytosine positions are detected by a primer extension reaction (Gonzalgo M L and Jones P A. (1997) Rapid quantitation of methylation differences at specific sites using methylation-sensitive single nucleotide primer extension (Ms-SNuPE). Nucleic Acids Res. 25:2529-31, WO 95/00669) or by enzymatic digestion (Xiong Z, Laird P W. (1997) COBRA: a sensitive and quantitative DNA methylation assay. Nucleic Acids Res. 25: 2532-4).

Another technique to detect hypermethylation is the so-called methylation specific PCR (MSP) (Herman J G, Graff J R, Myohanen S, Nelkin B D and Baylin S B. (1996), Methylation-specific PCR: a novel PCR assay for methylation status of CpG islands. Proc Natl Acad Sci USA. 93: 9821-6). The technique is based on the use of primers that differentiate between a methylated and a non-methylated sequence if applied after bisulfite treatment of said DNA sequence. The primer either contains a guanine at the position corresponding to the cytosine in which case it will after bisulfite treatment only bind if the position was methylated. Or the primer contains an adenine at the corresponding cytosine position and therefore only binds to said DNA sequence after bisulfite treatment if the cytosine was unmethylated and has hence been altered by the bisulfite treatment so that it hybridizes to adenine. With the use of these primers, amplicons can be produced specifically depending on the methylation status of a certain cytosine and will as such indicate its methylation state.

Another new technique is the detection of methylation via Taqman PCR, also known as MethyLight (WO 00/70090). With this technique it became feasible to determine the methylation state of single or of several positions directly during PCR, without having to analyse the PCR products in an additional step.

In addition, detection by hybridisation has also been described (Olek et al., WO 99/28498).

Further publications related to the use of the bisulfite technique for methylation detection in individual genes are: Grigg G, Clark S. (1994) Sequencing 5-methylcytosine residues in genomic DNA. Bioessays 16: 431-6; Zeschnigk M, Schmitz B, Dittrich B, Buiting K, Horsthemke B, Doerfler W. (1997) Imprinted segments in the human genome: different DNA methylation patterns in the Prader-Willi/Angelman syndrome region as determined by the genomic sequencing method. Hum Mol Genet. 6: 387-95; Feil R, Charlton J, Bird A P, Walter J, Reik W. (1994) Methylation analysis on individual chromosomes: improved protocol for bisulphite genomic sequencing. Nucleic Acids Res. 22: 695-6; Martin V, Ribieras S, Song-Wang X, R10 MC, Dante R. (1995) Genomic sequencing indicates a correlation between DNA hypomethylation in the 5′ region of the pS2 gene and its expression in human breast cancer cell lines. Gene 157 : 261-4; WO 97/46705, WO 95/15373 and WO 97/45560.

Elevated Levels of Circulating DNA

Another characteristic property of cancer and other cell proliferative diseases is an increased amount of free floating, circulating DNA in blood and/or serum. Also cell death caused by for example toxic doses of bacterial lipopolysaccharide, HgC12, CC14, cyclophosphamide and hydroxyurea triggers the release of products of chromatin catabolism, particularly of DNA into extracellular spaces. At least those have been shown to be responsible for the release of extracellular DNA in plasma in mice, in a dose dependent relationship. Hence it was suggested to use the quantitation of extracellular DNA for investigating in vivo cell death phenomena induced by toxic agents and drugs (Bret et al. (1990) Toxicology 61 (3): 283-92).

It is known that plasma DNA content reflects the amount of cell death occurring in the whole body and is increased during destructive pathological processes, including cancer. Increased DNA contents in serum have been found in correlation with Systemic Lupus Erythematosus (Leon et al. (1977) Cancer Res. 37: 646-650), malignant gastrointestinal disease (Shapiro et al. (1983), Cancer 51:2116-2120), pancreatic cancer (Anker et al. (1999), Cancer Metastasis Rev. 18: 65-73) and lung cancer (Maebo A. (1990), Jap J Thoraic Dis 28: 1085-1091 and Fournie et al. (1995), Cancer Let 2: 221-227). Whilst healthy human beings have free floating DNA levels in the range of 2-30 ng/ml, cancer patients, more specifically patients with Systemic Lupus Erythematosus in an early study from 1977 showed levels of up to 180 ng/ml (Leon et al. (1977) Cancer Res. 37: 646-650). In a publication from Jahr et al. a table is shown which describes plasma DNA levels from 23 patients grouped into 12 different tumour groups. In the most extreme case the DNA level was increased 100× compared to the mean value of DNA level in healthy patients. They concluded that elevated levels of circulating DNA appear to be a characteristic feature of most, but not all of the carcinoma diseases. The determined level of circulating DNA alone could not be correlated to the type of cancer or to the clinical status. But it has to be said that in Jahrs study no more than 4 repeats of any one tumour have been performed (Jahr et al. (2001), Cancer Res 61: 1659-1655).

Jahr et al. tried to analyse how much of this circulating DNA originates from tumour cells. It was reported from studies based on tumour specific microsatellite changes that nearly all the circulating plasma DNA originated from tumour cells (Goessl C. et al. (1998) Cancer Res., 58: 4728-4732). Other studies contrarily detected wild type DNA in the plasma of nearly all of the cancer patients. To be able to distinguish between tumour DNA and non tumour DNA they determined the DNA's methylation status, assuming that methylated DNA derived from the tumour tissue only and non methylated DNA from healthy cells. It was found that when the DNA count in the plasma was very high, the percentage of methylation was quite low, whereas when the DNA level was rather low the percentage of methylated DNA was—in one case at least—up to above 90%. The authors stress the fact that it would be difficult to investigate whether the unmethylated DNA originates from the neighbouring tumour tissue or from some other source “because DNA markers that distinguish defined cell types are not available”. In said paper evidence is discussed which supports the idea that the circulating DNA origins from apoptotic and necrotic cells.

Although the exact mechanism of the release of circulating DNA remains to be proved, an active release of circulating DNA from highly proliferating cells has also been proposed (Anker et al. (1999), Cancer Metastasis Rev. 18: 65-73). Herein the authors discuss why the origin of circulating DNA in the blood stream of cancer patients is most likely to be ‘active release’, rather than lysis of circulating cancer cells, necrosis or apoptosis.

Botezatu et al. described how to detect extracellular DNA in urine and how to analyse this DNA in order to diagnose cancer (Botezatu et al. (2000) Genetic analysis of DNA excreted in urine: a new approach for detecting specific genomic DNA sequences from dying cells in an organism. Clin Chem 46:1078-1084). Unlike previous work illustrating the diagnostic use of urine for cancer detection (Mao L. (1996) Genetic alterations as clonal markers for bladder cancer detection in urine. J Cell Biochem Suppl 25:191-196 and Eisenberger et al. (1999) Diagnosis of renal cancer by molecular urinalysis. J Natl Cancer Inst 91: 2028-2032), the cancer types chosen by Botezatu et al., namely pancreatic and colorectal carcinomas, are not of urologic origin. Previous work has indicated that pancreatic and colorectal cancer cells (Anker et al. (1997) K-ras mutations are found in DNA extracted from the plasma of patients with colorectal cancer. Gastroenterology 112: 1114-1120) can release tumoral DNA into the plasma. The new results by Botezatu et al. go one step further by suggesting that tumour DNA, following its release into the blood stream, will be excreted into the urine in sizes large enough for PCR analysis and hence applicable to our techniques on how to determine methylation patterns.

The data by Botezatu et al. include only patients with relatively advanced diseases (stages III and IV), the applicability of urine DNA analysis to the detection of early non-urologic malignancies remains to be demonstrated in future studies (Lo et al. (2000) Molecular Testing of Urine: Catching DNA on the Way Out. Clinical Chemistry 46: 1039-1040).

Methods on Quantitation of Nucleic Acids

Accurately determining the DNA concentrations of crude chromosomal or purified plasmid DNA samples is an essential step in quantitative manipulations of DNA. Two types of methods are widely used to measure the amount of nucleic acid in a preparation. If the sample is pure (i.e. without significant amounts of contaminants such as proteins, phenol, agarose or other nucleic acids), spectrophotometric measurement of the amount of ultraviolet irradiation absorbed by the bases is simple and accurate. Two different techniques rely on spectrophotometric and/or fluorometric analyses, for example to determine the concentration of a dilute sample of plasmid DNA purified by two passes through an ethidium bromide—caesium chloride (EtBr—CsCl) centrifugation gradient. The sample can either be tested on an for example LKB Biochrom Ultrospec II spectrophotometer for absorbance at wavelengths of 260 nm and 280 nm, or it can be tested for emission of 460 nm on the Hoefer TKO 100 mini-fluorometer in the presence of bisbenzimidazole, a fluorescent dye known as Hoechst H 33258 (manufactured by American Hoechst Corporation), that has an excitation maximum at 356 nm and an emission maximum of 458 when bound to DNA (Labarca and Paigen (1980) Anal. Biochem. 102, 344-352). The spectrophotometer detects absorbance due to RNA as well as DNA, while the Hoechst dye used in the fluorometer interacts specifically with adenosine and thymidine residues of DNA. Due to the highly specific nature of the Hoechst dye the mini-fluorometer seems to be most accurate for quantitation of crude chromosomal DNA, but less reliable for plasmids and other DNA of limited complexity.

If the amount of DNA or RNA is very small or if the sample contains significant quantities of impurities, the amount of nucleic acid can be estimated from the intensity of fluorescence emitted by ethidium bromide molecules intercalated into the DNA (Sambrook; Fritsch and Maniatis (1989) Molecular Cloning—A laboratory manual (second edition) 3: E.5). A simple application of this general approach is the use of EtBr agarose plates. DNA samples of 2-10 ul are spotted onto 1% agarose containing 0.5 ug/ml EtBr within a Petri dish. Afterwards, the plate is exposed to UV light and photographed. Another variation is to mix 5-10 ul of a 0.5 ug/ml solution of EtBr with 10 ul of DNA spotted onto plastic film wrap or a siliconised glass slide placed on top of a UV transilluminator. The advantage of this method is that DNA samples with as little as 1-10 ng of DNA can be quantitated within minutes. The disadvantage is the intercalation of the dye with RNA as well as DNA and its limitation to double stranded DNA.

Other methods for quantitating DNA are for example, Invitrogen's nucleic acid quantitation DNA Dipstick [TM] kit, which is claimed to be sensitive enough to detect as little as 0.1 ng/ul of nucleic acid. Unfortunately, the method cannot be used with samples containing more than 10 ng/ul of nucleic acids. (see: Trends in Biochemical Sciences 19, 46-47).

Methods on Detection of Specific DNA

Methods to detect and quantify specific nucleic acids are used in detecting microorganisms, viruses and biological molecules. Hence they are used in human and veterinary medicine, food processing and environmental testing. Additionally, the detection and/or quantification of specific biomolecules from biological samples (e.g. tissue, sputum, urine, blood, semen, saliva) has applications in forensic science, such as the identification and exclusion of criminal suspects and paternity testing as well as medical diagnostics. However the majority of such methods is based on two techniques: hybridisation and PCR. Both of which detect and quantify a certain specific part of the genomic DNA.

Hybridisation is known as one of the methods to detect a nucleic acid having a specified base sequence (hereafter referred to as “target nucleic acid”). This method employs an oligonucleotide probe having a base sequence capable of hybridising to the target nucleic acid as a detection probe to form a hybrid, and performs detection of the target nucleic acid by detecting the hybrid through various detection means.

In patent U.S. Pat. No. 6,228,592 the drawbacks of this technique get mentioned, especially when trying to apply those to detecting a specific sequence in a surrounding environment like a biologically active fluid or especially in a living cell. When a detection probe is introduced into the cytoplasm, it will a) rapidly move to the nucleus and b) the probe or the hybrid between the detection probe and the target nucleic acid is rapidly digested by various kinds of nuclease existing in the cytoplasm, which renders the detection of the target nucleic acid difficult. This can be circumvented by using an oligonucleotide probe having a base sequence capable of hybridising to the specified base sequence of a target nucleic acid, which is bound to a nuclear membrane unpermeable molecule via a linker and labelled with a fluorescent dye; forming a hybrid between the target nucleic acid and the probe. A change in fluorescence of the fluorescent dye due to formation of the hybrid thereby detects the existence of the target nucleic acid in the cytoplasm of a living cell or any other background contaminated with DNAses.

Another type of process for the detection of hybridised nucleic acid takes advantage of the polymerase chain reaction (PCR). The PCR process is well known in the art (U.S. Pat. Nos. 4,683,195, 4,683,202, and 4,800,159). To briefly summarise PCR, nucleic acid primers, complementary to opposite strands of a nucleic acid amplification target sequence, are permitted to anneal to the denatured sample. A DNA polymerase (typically heat stable) extends the DNA duplex from the hybridised primer. The process is repeated to amplify the nucleic acid target. If the nucleic acid primers do not hybridise to the sample, then there is no corresponding amplified PCR product. In this case, the PCR primer acts as a hybridisation probe. PCR-based methods are of limited use for the detection of nucleic acid of unknown sequence.

In a PCR method, the amplified nucleic acid product may be detected in a number of ways, e.g. incorporation of a labelled nucleotide into the amplified strand by using labelled primers. Primers used in PCR have been labelled with radioactivity, fluorescent dyes, digoxygenin, horseradish peroxidase, alkaline phosphatase, acridinium esters, biotin and jack bean urease. PCR products made with unlabeled primers may be detected in other ways, such as electrophoretic gel separation followed by dye-based visualisation.

Fluorescence techniques are also known for the detection of nucleic acid hybrids. U.S. Pat. No. 5,691,146 describes the use of fluorescent hybridisation probes that are fluorescence-quenched unless they are hybridised to the target nucleic acid sequence. U.S. Pat. No. 5,723,591 describes fluorescent hybridisation probes that are fluorescence-quenched until hybridised to the target nucleic acid sequence, or until the probe is digested. Such techniques provide information about the existence of a target that hybridises to said probes, and are of varying degrees of usefulness for the determination of single base variances in sequences. Some fluorescence techniques involve digestion of a nucleic acid hybrid in a 5′ to 3′ direction to release a fluorescent signal from proximity to a fluorescence quencher, for example, TaqMan^{RTM} (Perkin Elmer; U.S. Pat. Nos. 5,691,146 and 5,876,930).

Real Time PCR:

Real time PCR monitoring using fluorescence has been described in several manners. Firstly, the binding of double stranded DNA specific fluorescent dyes such as ethidium bromide allows for the monitoring of the accumulation of PCR product by correlation with increased fluorescence. A second detection method, polymerase mediated exonuclease cleavage utilises the 5′ exonuclease activity of polymerases such as Taq. An oligonucleotide probe that is complementary to the PCR product, yet distinct from the PCR primer is labeled with a FRET pair such that the donor molecule is quenched by an acceptor molecule. During PCR amplification, the 5′ exonuclease proceeds to digest the probe, separating the FRET pair and leading to increased fluorescence. A variation on this technology uses a nucleic acid wherein the FRET pair is internally quenched, for example, by having a hairpin conformation. Upon hybridisation to a sequence of interest, the FRET pair is separated and the donor molecule emits fluorescence. This technology can be used, for example, for the analysis of SNPs.

An alternative technology is based on the use of two species of hybridisation probes, each labelled with a member of a FRET pair. Upon hybridisation of both probes to the target sequence in adequate proximity, a fluorescent signal is emitted. Again, this technology may be used for the detection of SNPs.

A major advantage of the use of such FRET based PCR technologies is that the reaction may be monitored in a closed tube reaction, suitable for use in high and medium throughput and reducing the probability of contamination.

Methods on Extracting and Detecting DNA in Bodily Fluids

Methods for the detection of circulating DNA are described in a number of articles. In the majority of cases for separating the DNA from the biological sample scientists rely on a kit supplied by Qiagen, called QIAamp Blood Kit (Qiagen, Hilden, Germany):

For example in Jahr et al. (2001), Cancer Res 61: 1659-1655: “After having separated the plasma from blood cells by centrifugation at 3000 g for 20 min the DNA from the blood plasma can be extracted using the QIAamp Blood Kit (Qiagen, Hilden, Germany) using the blood and body fluid protocol referring to Wong et al. (1999), Cancer Res 59: 71-73 and Lo et al. (1998) Am. J. Genet. 62: 768-775.”

Wong et al. (1999), Cancer Res 59: 71-73: “Blood samples are centrifuged at 3000 g and plasma and serum are carefully removed from the EDTA-containing and plain tubes, respectively, and transferred into plain polypropylene tubes. The buffy coat fraction from the EDTA-containing tubes was also collected to study the presence of circulating tumour cells in the peripheral blood. The samples were stored at −70 C or −20 C until further processing. DNA from plasma and serum samples was extracted using a QIAamp Blood Kit (Qiagen, Hilden, Germany) using the blood and body fluid protocol as recommended by the manufacturer (Chen et al. (1996). Microsatellite alterations in plasma DNA of small cell lung cancer patients. Nat Med 2: 1033-1035).”

Chen et al.: “Fresh frozen tissue was treated with SDS and proteinase K followed by phenol and chloroform extraction. Paraffin-embedded tissue was scraped from the slides and washed in xylol to remove paraffin. After the addition of one volume of ethanol, the mixture was centrifuged, and the pellet was digested with proteinase K and SDS, followed by phenol and chloroform extraction. Control Lymphocyte and plasma DNA were purified on Qiagen columns (Qiamp Blood Kit, Basel, Switzerland) according to the “blood and body fluid protocol”. Plasma (1-3 ml) was passed on the same column. After purification, 1 ml of plasma yields an average of 39 ng of DNA.

The amounts of plasma DNA can be determined by competitive PCR according to the method of Diviacco et al. (1992) Gene 122: 313-320, using for example the Lamin B2 locus as a typical example for a single copy gene. The competitor molecule carrying a 20-bp insert was obtained directly from two amplification products by the overlap extension method (Diviacco et al. (1992) Gene 122: 313-320).

Quantitation of competitive templates can be obtained by OD260 measurement. A fixed amount of plasma DNA can be mixed with increasing amounts of the competitor template. For competitive PCR, two additional primers need to be designed. After PCR amplification and PAGE, two products are evidently corresponding to genomic and competitor templates. The ratios of the amplified products precisely reflect the initial concentration of genomic DNA versus that of the added competitor. Quantitation of competitor and genomic bands can be obtained by densitometric scanning of the ethidium bromide stained gel.

The results obtained by means of competitive PCR can be confirmed by quantitation with the control Kit DNA in the LightCycler System (Roche Diagnostics) using the LightCycler Control Kit DNA to amplify a 110 bp of the human Beta-globin gene. The amplicon can be detected by fluorescence using a specific pair of hybridisation probes (LC-Red 640).

A similar approach was used by Lee et al. to quantify genomic DNA of serum and plasma samples DNA by using reagents from an HIV assay kit (HIV Monitor Assay, Roche Molecular Systems, Emeryville, Calif.). Immediately after thawing, plasma and serum samples were microcentrifuged at maximum speed (Microfuge II, Beckman Instruments) for 5 minutes to produce clean plasma or serum, free aggregates and non-specific precipitates. Plasma and/or serum (100 ul) was removed and deposited into a 1.5 ml microcentrifuge tube containing 300 ul of working lysis reagent. The tube was then agitated vigorously for 3-5 seconds and incubated at room temperature for 10-15 minutes. After incubation, 400 ul of 100-percent isopropanol was added into each tube, which was then agitated for 3-5 seconds and microcentrifuged at 10000 g (12000 rpm Microfuge II, Beckman Instruments) for 15 to 30 minutes at room temperatures. Supernatant was removed and 1 ml of 70 percent ethanol was added to each tube; these steps were followed by microcentrifugation at 10.000 g for 5-10 minutes at room temperature. The supernatant was removed and then the DNA pellets were left overnight at room temperature to evaporate any remaining ethanol. The pellet was resuspended in 100 ul of PCR solution A (100 mM KCl, 10 mM Tris, 2.5 mM MgCl₂; pH 8.3) and PCR solution B (10 mM Tris, 2.5 mM MgCl₂, 1% Tween-20, 1% Nonidet P-40; pH 8.3).

Purified DNA was amplified with HLA DQ-alpha primers or human Y-chromosome primers. Standard curves were prepared and for quantification included in each amplification (Lee et al. (2001) Transfusion 41: 276-282).

In the patent U.S. Pat. No. 6,156,504 (Gocke et al.), which also relates to the detection of tumour-associated extracellular nucleic acid in plasma or serum fractions, an overview is given on several methods on how to extract and detect circulating DNA in blood and serum samples.

To determine the DNA concentration in a urine sample the samples need to be fresh, because human urine contains a nuclease activity (Botezatu et al. 2000). The fresh samples are centrifuged 10 min at 800 g and DNA is isolated from the supernatant as described by Labarca and Paigen (Labarca and Paigen (1980) Anal Biochem 102: 344-352).

It is also known in the art, that that human stool samples contain DNA derived from gastrointestinal cells. Said DNA can be utilised to test for the absence or presence of a specific kind of K-ras gene mutation, which allows to conclude whether the donor is likely to have developed a colon cancer.

Problem and Solution

In summary, the state of the art is to develop more and more nucleic acid based assays in order to detect the presence or absence of tumour indicating protein or cDNA of tumour related genes, so called tumour marker genes in blood or other bodily fluids. The detection of cancer specific alterations of genes involved in carcinogenesis, like oncogene mutations or deletions, tumour suppressor gene mutations or deletions, or microsatellite alterations will then allow a prediction of the patient to carry a tumour or not (for example patent WO 95/16792 or U.S. Pat. No. 5,952,170 to Stroun et al.). In an advanced stage the aim will be to produce a kit that allows the scientist to screen plenty of samples in little time with high accuracy. These kits will not only be of interest for an improved preventive medicine and early detection of cancer but also to monitor a tumours performance after therapy.

Also the detection of hypermethylation of certain genes, especially of certain promoter regions, has been recognised as an important indicator for the presence or absence of a tumour. To our knowledge so far all studies that dealt with methylation analysis looked at the methylation status of certain tumour marker genes, only. These genes are known to play a role in the regulation of carcinogenesis or in other words are believed to determine the switching on and off of tumorigenesis. Most advanced is the knowledge about methylation and prostate cancer. Hence a method employing the methylation analysis of a certain marker gene (GSTP1) indicating prostate cancer using DNA from a bodily fluid has been patented (U.S. Pat. No. 5,552,277). The determination of the methylation state of certain, yet to be identified indicator genes might even become a useful tool to predict the responsiveness of a patient towards chemotherapy and radiotherapy (Hanna et al. (2001) Cancer Res 61: 2376-2380). However, all those screening approaches are limited to certain cancer types. This is because they are all limited in that they look for certain marker genes, which are highly specific for a kind of cancer when found in a specific kind of bodily fluid. Another example is described by Usadel et al. They described that they could detect a tumour specific methylation pattern in the promoter region of a gene, called adenomatous polyopsis coli (APC) in serum samples of lung cancer patients but that no methylated APC promoter DNA could be detected in serum samples of healthy donors (Usadel et al. (2002) Cancer Research 6, 371-375). Therefore this marker qualifies as a good indicator for lung cancer and could be used specifically for the screening of people that have been diagnosed with lung cancer or maybe the monitoring of patients after surgical removal of a tumour for developing metastases in their lung. However, Usadel also describes that epigenetic alterations of the gene APC are common events in gastrointestinal tumour development, hence a blood screen with APC as a tumour marker only indicates the patient is developing a tumour, but it is unknown where that tumour would be located. This does not enable the physician to directly use this information to follow up with more detailed diagnosis or even treatment of the respective medical condition, as most of the available diagnostic or therapeutic measures will be specific for the organ involved. Especially if the lesion is still small in size, it will be very difficult for the physician to find out to which organ further diagnostics and therapies should be targeted. Therefore, although the cancer marker being present indicates that any treatment out of a large regimen of possibilities will be required, it does not help physician and patient at all in their decision on how to deal with this information. The physician would have to further investigate all possible organs for the presence of the lesion, and even if he finds something it is unclear whether the lesion is confined only to this specific organ. This is one of the problems in the state of the art that embodiments of the present invention will solve by providing information about the disease that is organ specific.

The invention described in patent U.S. Pat. No. 6,156,504 (Gocke et al.) also relates to the detection of extracellular nucleic acid in plasma or serum fractions, but the patent only covers a method to detect mutated extracellular K-ras nucleic acid in blood. This is another example for the dependence of most assays on specific tumour marker genes, in this case the oncogene K-ras. For a number of cancer types though specific genes are not even known yet.

Typically “cancer markers” indicate the existence of a tumour or other cell proliferative disease but they are not specific for a certain kind of tissue. A typical cancer marker detects the likelihood to have developed one kind of tumour out of a group of different possible tumours, but without allowing conclusions as to the specific type of tumour or the organ of origin. So the tumour marker is specific for detecting tumours but not specific for the tissue, organ or cell type.

Therefore in an early screen, when there is no reason to assume that the patient suffers from a specific kind of cancer, a screen with tumour specific markers, that are unable to predict the location of the tumour would not be of much help to the patient, who would now have to wait for results from other diagnostic tests screening his whole body.

On the other hand, if there were tumour markers that were specific for one kind of tumour, tailoring the diagnosis to a specific location or a specific tissue type affected, a very early screen would require to simultaneously test for every possible cancer specific gene alteration known so far. This can be regarded as unfeasible.

Cell proliferative diseases cause extracellular DNA levels in blood and in other bodily fluids to rise. To our knowledge the quantitation of extracellular DNA in humans has never been used to predict the risk of a patient to carry a cell proliferative disease like for example cancer. Some reports have been published where elevated levels of circulating DNA in blood of cancer patients are mentioned, but these were solely utilised as a source for easier accessible DNA in order to analyse its properties further (Jahr et al. 2001). It is also known that these DNA molecules origin from the tissue where the cells are dying, for whatever reason (as discussed above). Nevertheless, up to now there has been a lack of know-how in order to determine the DNAs origin and therefore it wasn't possible to link the general result of increased DNA levels in a bodily fluid like blood to the risk of a cell proliferative disease in a specific organ. This lack has been due to the unavailability of tissue specific markers (Jahr et al. 2001), which would allow a determination of the DNAs origin. This is exactly the gap that this invention is able to close.

Genes, which could serve as tissue, cell type or organ specific markers have been described (for example Adorjan et al. “Tumour class prediction and discovery by microarray-based DNA methylation analysis” (2002) Nucleic Acids Res. 30, e21). It is also known that certain medical conditions, such as cell proliferative or inflammatory diseases, cause the level of free floating DNA in a bodily fluid to increase. However, the idea to employ those markers to determine the source of said free floating DNA and to hereby enable a fast and accurate diagnostic test for the detection of organs, tissues or cell types that suffer from such a medical condition is a significant advantage to the state of the art.

Wherein so far marker genes have been used for an early diagnosis of certain medical conditions this has only been done with a specific medical condition in mind. For example, a patient suspicious of having developed a colon cancer can have his stool sample tested with a cancer marker like K-ras. A patient suspicious of having developed a prostate cancer can have his ejaculate sample tested for a prostate cancer marker like GSTPi. However, for a general screen wherein the patient has no specific suspicion as to which organ or tissue might develop a cell proliferative disease or similar (for example, an individual who has accidentally been exposed to a high amount of radiation), there is to our knowledge no method described in the prior art on how to detect such a medical condition in a fast and accurate way.

Although it has been described that elevated free floating DNA levels in blood are indicative of cancer or other cell proliferative diseases, detecting the level of free floating DNA does not on its own serve as a useful diagnostic method as the information gained is too unspecific to be of any use.

However, when determining where said free floating DNA originates from, which so far has not been described, the diagnostic value of such an assay increases dramatically. This is because such an assay elucidates the location of said DNA and the possible cause. That way an early screen that does reveal the organ, tissue or cell type affected by a cell proliferative disease is highly advantageous. The information gained will aid the further diagnostic procedure. It tells the practitioner quite precisely what the next steps towards a more differentiated diagnosis would need to be and gives guidance as to which clinical specialist to refer the patient to.

It is therefore an object of the present invention to provide a method that enables a prediction that the patient is likely to suffer from a medical condition, for example a cell proliferative disease in a specified organ, tissue or cell type, by determining the tissue, organ or cell type that releases a significant part of the free floating, circulating DNA in the patient's blood or other bodily fluid.

### BRIEF DESCRIPTION OF THE INVENTION

The present invention provides a method for the analysis of circulating, free floating nucleic acids in bodily fluids. It discloses a means on how to predict which organ, tissue or cell type has developed a medical condition, by employing means of distinguishing between DNA originating from different healthy or different diseased tissues, organs or cell types of the human body. Characteristic methylation patterns of certain genes can be positively correlated with specific organs, tissues and cell types. Preferably the identification of the free floating DNA's origin, or in other words the determination of the organic source of a significant part of those circulating nucleic acids in said bodily fluid is done by an assay that detects methylation at specific CpG sites. It is especially preferred, to detect methylation by nucleic acid based methods, such as hybridization, sequencing and PCR, or even more preferably, by employing real-time PCR methods. The result of said analysis give further guidance to a practitioner on how to tailor a more differentiated diagnostic strategy.

### DETAILED DESCRIPTION OF THE INVENTION

‘Bodily fluid’ herein refers to a mixture of macromolecules obtained from an organism. This includes, but is not limited to, blood, blood plasma, blood serum, urine, sputum, ejaculate, semen, tears, sweat, saliva, lymph fluid, bronchial lavage, pleural effusion, peritoneal fluid, meningal fluid, amniotic fluid, glandular fluid, fine needle aspirates, nipple aspirate fluid, spinal fluid, conjunctival fluid, vaginal fluid, duodenal juice, pancreatic juice, bile and cerebrospinal fluid. This also includes experimentally separated fractions of all of the preceding. ‘Bodily fluid’ also includes solutions or mixtures containing homogenised solid material, such as faeces.

A ‘methyl-specific agent’ herein refers to any chemical or enzyme interacting or reacting with nucleic acids in such a way that a methylated and a non-methylated nucleobase react differently, resulting in differently modified nucleobases. By acting specifically on either the one or the other or by interacting with both in a different way it will be easier, by methods available today, to differentiate between these nucleobases than it has been before the interaction with said ‘methyl-specific agents’. Examples for treatment with a ‘methyl-specific agent’ are the so called ‘bisulfite treatment’ or treatment with methylation sensitive restriction enzymes. Throughout the document the treatment will also be referred to as ‘chemical pretreatment’.

The term ‘bisulfite treatment’ refers to the method commonly known to the person skilled in the art. Examples for the treatment can be found, for example, in several of the references cited herein.

The term ‘free floating DNA’ in general is to be understood to relate to extracellular deoxynucleic acids, for example unbound DNA or circulating nucleic acids as present in bodily fluids as defined above. The DNA can, nevertheless, be bound to proteins in said bodily fluid, this will also be understood as “free floating” in the context of the present invention. In some rare instances, as, for example, the analysis of DNA that is derived from single cells or clumps of cells that are derived from organs or tissues (e.g. lung cells that are expectorated) and that are present in the bodily fluid to be analysed, the cells have to be broken up in order to release their DNA. The DNA that is released from these cells in said bodily fluid will also be understood as “free floating” in the context of the present invention.

In the context of the present invention, the term ‘hybridisation’ is to be understood as a bond of an oligonucleotide to a completely complementary sequence along the lines of the Watson-Crick base pairings in the sample DNA, forming a duplex structure.

In the context of the present invention, the term ‘an essential fraction’ is to be understood as a part of a mixture of compounds (e.g. total DNA in a bodily fluid) that represents a qualitative (or statistic) fraction of the whole mixture, in contrast to a quantitative fraction. In other words ‘an essential fraction’ is a small amount of the total DNA that, nevertheless, reflects the statistic distribution of the different DNA molecules in said total DNA.

The present invention provides a method for detecting the presence or absence of a medical condition in a tissue, cell type or organ of an individual, comprising the following steps a) retrieving a bodily fluid sample from said individual, b) determining the amount or presence (detectable above a given threshold) of free floating DNA that originates from said tissue, cell type or organ in said sample and c) determining the presence or absence of a medical condition based on the amount or presence (detectable above a given threshold) of free floating DNA that originates from said tissue, cell type or organ.

The present invention provides a method to determine the presence or absence of a medical condition such as inflammatory diseases or cell proliferative diseases, and in particular cancer. The method employs several steps starting with the retrieval of an individual's sample in form of a tissue sample or a biological fluid like blood, serum, urine or other fluids as defined above. The second step is the determination of the organ, tissue or cell type that a significant portion of said floating DNA is derived from. Said determination of the amount or presence (detectable above a given threshold) of free floating DNA that originates from a specific organ, tissue or cell type is done by determining specific characteristics of the free floating DNA and comparing it with the characteristics of DNA originating from a specific organ, tissue or cell type. From this determination the presence or absence of a medical condition can be concluded upon. Hence the third step is the determination of the presence or absence of a medical condition based on the amount or presence (detectable above a given threshold) of free floating DNA that originates from said organ, tissue or cell type.

Knowing the correlation of specific organs, tissues or cell types with specific characteristics of for example marker genes or marker sequences the origin of said significant portion of free floating DNA can be determined.

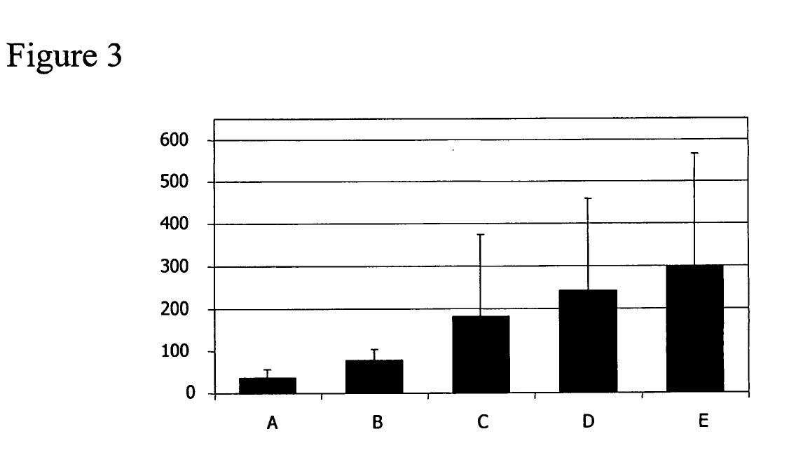

In a specific embodiment the method additionally employs the step of determining the amount of total free floating DNA in said sample. This allows the prediction of the likelihood that said individual develops a disease, wherein the disease is specified by showing increased levels of total free floating DNA in a bodily fluid as defined above. An increased level of the total free floating DNA is understood to be a free floating DNA level significantly higher than the average level in the bodily fluid of a healthy person (which is to be determined in a series of experiments, but will for example in serum samples likely be specified somewhere between 10 and 100 ng/ml), and herein allowing to perform an informative DNA methylation analysis. The correlation between an increased level of DNA in a bodily fluid and the absence or presence of a disease like cancer can be seen at FIG. 3, which shows results from our own studies.

In another preferred embodiment the amount of DNA originating from a specific organ, tissue or cell type is also quantified, allowing to compare said fraction with the total amount of free floating DNA and concluding from this ratio on the absence or presence of a medical condition.

In a preferred embodiment said medical condition is a cell proliferative and/or neoplastic disease. It is especially preferred that said medical condition is a type of cancer.

The knowledge achieved allows to predict if the individual carries a medical condition, such as a cell proliferative disease in said tissue, organ or cell type. For example, a patient with a substantial amount of free floating DNA originating from liver, might have developed a liver tumour. To validate this, the next step could be to employ, for example, a tailored test assay for disease indicating marker gene expression, specific for said organ or tissue.

In a particularly preferred embodiment, the characteristics of said free floating DNA that are specific for a certain organ, tissue or cell type are characterised as being specific methylation statuses of certain marker genes or nucleic acids. The determination of the origin of said free floating DNA is based on a methylation pattern analysis of said DNA captured from said sample. The method is based upon the determination of the tissue that contributes significantly to the total amount of the free floating DNA in said biological fluid, by detecting tissue specific methylation patterns on said free floating DNA. It is therefore a preferred embodiment of the invention that said method is characterised in that the amount or presence (detectable above a given threshold) of DNA originating from a certain organ or tissue is determined by analysing a DNA methylation pattern that is characteristic for said organ, tissue or cell type. It is especially preferred that said methylation pattern is not found in other organs tissues or cell types involved in the medical condition of interest. This is because when, for example, analysing the free floating DNA of an individual who has been diagnosed with liver cancer, a test might reveal whether the cancer has spread to his kidneys or not when testing urine samples, as urine normally will contain only very small amounts of transrenal DNA from liver cells. In that case the methylation pattern must be differential between liver cells and any urinary tract cells. However, the methylation pattern does not need to be specific as to also exclude organs like lung, for example.

Said method is independent of the use of so called tumour markers. Wherein tumour markers are understood to be genes or nucleic acids that show measurable specific characteristics when isolated from a tumour cell as in opposite to a healthy cell, tissue markers are understood to be genes or nucleic acids that show measurable specific characteristics for the specific tissue (organ or cell type) they are isolated from. The presence of tissue specific markers or an increased amount thereof in a body fluid where these normally cannot be found or at a lower level, is indicative of a disease being present in that specific tissue, without the need for a disease specific marker, e.g. a tumour marker.

Tissue specific methylation patterns can be determined by analysis of the methylation statuses of either single genes or sets of genes, which will show differentially methylated CpG positions according to the specific organ, tissue or cell type they originate from. Preferably the analysis of said tissue, organ or cell-type specific methylation patterns on the circulating nucleic acids in said bodily fluid is done by an assay that detects methylation at specific CpG sites by restriction enzyme analysis. It is especially preferred however, to detect methylation by nucleic acid based methods, such as hybridisation, sequencing and PCR, or even more preferably, by employing real-time PCR methods.

It is a preferred embodiment that said method is characterised in that the methylation pattern is determined by subjecting the free floating DNA to a chemical or enzymatic treatment that converts all unmethylated cytosines in the DNA into uracil but leaving position 5-methylated cytosines unchanged.

In a especially preferred embodiment said treatment is the ‘bisulfite treatment’. It is further preferred that said DNA is isolated prior to said treatment.

It is also a preferred embodiment of this invention that the method according to the present invention is characterised in that said bodily fluid sample is conditioned prior to determining the amount of total free floating DNA or determining the amount or presence (detectable above a given threshold) of free floating DNA originating from a specific organ, tissue or cell type.

The invention hereby provides a means for the improved diagnosis, prognosis, staging and grading of cancer, at a molecular level, by employing the capacity to differentiate between sources of free floating DNA in bodily fluids. Said capacity can also be used to discover the actual reason for the increase of nucleic acids in a bodily fluid, such as blood or serum.

Furthermore, the disclosed invention provides improvements over the state of the art in that current methods of diagnosing, prognosing, staging and grading of cancer, are mainly based on histological and cytological analyses that require a biopsy that provides a sufficient amount of tissue. Also, methylation analysis technology until recently required amounts of DNA that could only be provided by biopsy samples. Only since it has become possible to perform methylation analysis on as little amount of DNA as there is in a bodily fluid sample for example, by Real-Time PCR (Usadel et al. Cancer Research 62, 371-375), the described method has become feasible. Therefore, the method according to the present invention can be used for classification of easily accessible samples like bodily fluids that make a biopsy avoidable.

The present invention further makes available a method for ascertaining genetic and/or epigenetic parameters of genomic DNA.

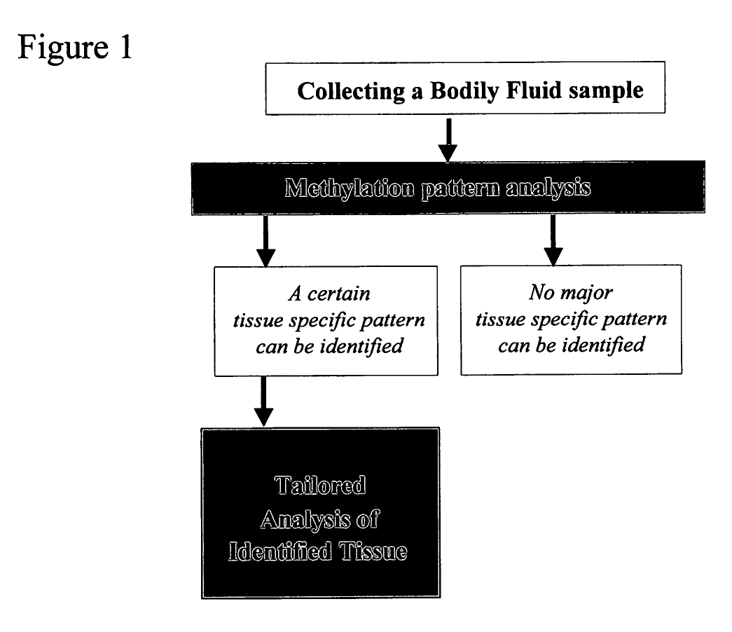

The method is described in more detail now. The method comprises of the following steps, which are described with reference to FIG. 1 that shows a flow chart of the method according to the present invention:

In the first step of the method, a sample is retrieved from a patient or individual in form of said bodily fluids (as defined above). The retrieval of the said sample can be done in any way known to a person skilled in the art. The detailed description can be found in relevant technical articles and text books that describe the state of the art. This includes but is not limited to ventricular puncture, also known as CSF collection, a procedure to obtain a specimen of cerebrospinal fluid (CSF); thoracentesis, referring to inserting a needle between the ribs into the chest cavity, using a local anaesthetic to obtain the pleural effusion fluid; amniocentesis, referring to a procedure performed by inserting a hollow needle through the abdominal wall into the uterus and withdrawing a small amount of fluid from the sac surrounding the foetus; but also urine, sperm and sputum collection.

In a preferred embodiment the samples are obtained from any bodily fluids as mentioned in the definition above. In a further and especially preferred embodiment the samples are obtained from whole blood, blood serum, urine, saliva or ejaculate from said individual.

In the second step the amount or presence (detectable above a given threshold) of free floating DNA in said sample that originates from a specific tissue, organ or cell type is determined. However, in a preferred embodiment the sample is conditioned prior to this step. Therefore before describing step 2 said conditioning is described in more detail first. However the following steps are also enabled without doing any of the treatment described as conditioning now:

The free floating nucleic acids may be extracted and/or separated from RNA if necessary. However the following steps are also enabled without doing any of the aforementioned treatment. Also, the DNA may be purified, or otherwise conditioned and prepared, before determination of the source of said DNA or before quantification of it. Purification may be done for example on Qiagen columns supplied in the Qiamp Blood Kit as described (Chen et al. (1996) Nature medicine 2, 1033-1035). The quantitation may take place either immediately after retrieval of the sample or after an unspecified time of storage of said sample. In a preferred embodiment of the method the free floating DNA will be separated from the cell bound DNA via centrifugation either after the amount of total DNA in said sample (including the cell bound) has been determined or without determining the cell bound DNA at all.

Any process mentioned in said optional step of conditioning may be done by means that are standard to one skilled in the art, these include the use of detergent lysates, sonification and vortexing with glass beads.

In a preferred embodiment the sample is also conditioned by means of preservation, like heating or adding chemicals to deactivate or inhibit deoxyribonucleases or other nucleic acid degrading enzymes; storage at reduced (below room temperature) or not reduced temperatures; cooling; heating; the addition of detergents; filtering and/or centrifugation. For example the sample may be treated with proteinase K (from Boehringer Mannheim) and sodium dodecyl sulfate at 48° C. overnight before separating out the DNA as described (Eisenberger et al (1999) J Natl Cancer Inst 91: 2028-2032) for serum samples.

Also conditioning in this context comprises applying methods to concentrate the DNA in said sample. These methods can be either one or several of the methods mentioned in the description of prior art and may be any by means that are standard to one skilled in the art. Some of those are described in detail in Appendix E of the well known lab manual Sambrook, Fritsch and Maniatis (1989) Molecular Cloning—A Laboratory Manual (second edition): precipitation of DNA in microfuge tubes, precipitation of RNA with ethanol, concentrating nucleic acids by extraction with butanol (vol 2: E. 12, E.15 and E. 16 respectively).

In preferred embodiments conditioning can also mean any kind of chemical treatment, like adding an anti-coagulant, treatment with reducing agents, treatment with intercalating chemicals or chemicals that build covalent bonds with the DNA.

In a preferred embodiment the DNA may be cleaved prior to the chemical treatment, this may be by any means standard in the state of the art, in particular with restriction endonucleases.

In the second step of the method, the methylation pattern of the free floating DNA is determined in order to discover where a significant amount of said DNA origins from.

It is preferred that said nucleic acid sample is first treated with a ‘methyl-specific agent’ like, but not limited to, bisulfite or with, for example, methylation sensitive restriction enzymes. In a preferred embodiment the extracellullar nucleic acids are chemically treated in such a manner that cytosine bases which are unmethylated at the 5′-position are converted to uracil, thymine, or another base which is dissimilar to cytosine in terms of hybridisation behaviour. This will be understood as treatment with a ‘methyl-specific agent’ or as ‘chemical pre-treatment’. Said chemical conversion may take place in any format standard in the art. This includes but is not limited to modification within agarose gel or in denaturing solvents. The nucleic acid may be, but doesn't have to be, concentrated and/or otherwise conditioned before the said nucleic acid sample is treated with said agent. In this second step of the method, it is preferred that the above described treatment of extracellular nucleic acids is carried out with bisulfite (sulfite, disulfite) and subsequent alkaline hydrolysis, which results in a conversion of non-methylated cytosine nucleobases to uracil or to another base which is dissimilar to cytosine in terms of base pairing behaviour.

The double stranded DNA is preferentially denatured. This may take the form of a heat denaturation carried out at variable temperatures. The denaturation temperature is generally depending on the buffer but for high molecular weight DNA it can be as high as 90° C. However, the analysis may be upon smaller fragments which do not require such high temperatures. In addition as the reaction proceeds and the cytosine residues are converted to uracil the complementarity between the strands decreases. Therefore, a cyclic reaction protocol may consist of variable denaturation temperatures.

The bisulfite conversion then consists of two important steps, the sulfonation of the cytosine and the subsequent deamination. The equilibra of the reaction are on the correct side at two different temperatures for each stage of the reaction. The temperatures and length at which each stage is carried out may be varied according to the specific requirement of the situation. However, a preferred variant of the method comprises a change of temperature from 4° C. (10 minutes) to 50° C. (20 minutes). This form of bisulfite treatment is state of the art with reference to WO 99/28498.

It is preferred that sodium bisulfite is used as described in WO 02/072880. Especially preferred is the so called agarose bead method, wherein the DNA is enclosed in a matrix of agarose, thereby preventing the diffusion and renaturation of the DNA (bisulfite only reacts with single-stranded DNA), and replacing all precipitation and purification steps with fast dialysis (Olek A, et al., A modified and improved method for bisulfite based cytosine methylation analysis, Nucleic Acids Res. 24:5064-6, 1996). It is further preferred that the bisulfite treatment is carried out in the presence of a radical trap or DNA denaturing agent, such as oligoethylenglykoldialkylether or preferably Dioxan.

Said chemical conversion may take place in any format standard in the art. This includes but is not limited to modification within agarose gel, in denaturing solvents or within capillaries.

In preferred embodiments bisulfite conversion within agarose gel will be done as described by Olek et al., Nucl. Acids. Res. 1996, 24, 5064-5066. The DNA fragment is embedded in agarose gel and the conversion of cytosine to uracil takes place with hydrogensulfite and a radical scavenger. The DNA may then be amplified without need for further purification steps.

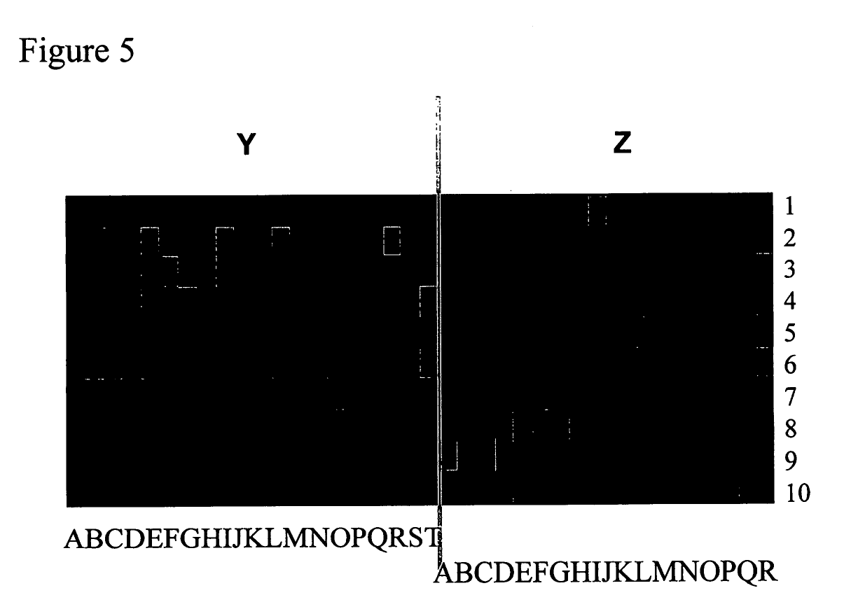

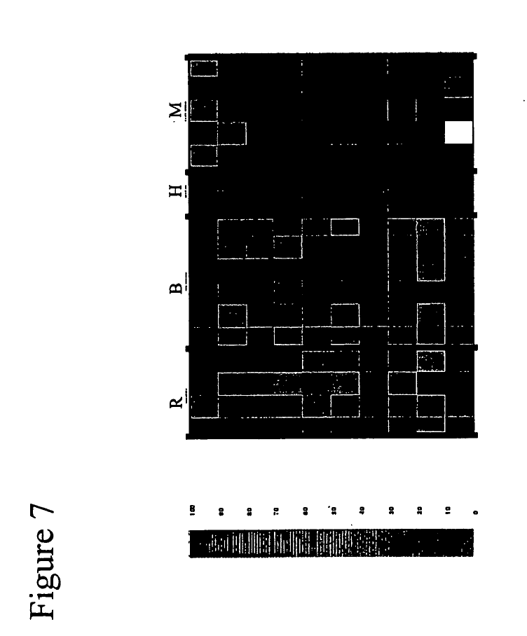

If a CpG positions is only ever specifically methylated when the corresponding DNA sequence was isolated from one cell type, for example, kidney cells but said CpG position is not methylated when the DNA was isolated from another cell type, for example, liver cells, blood cells, bladder cells or colon cells etc. said CpG position is an ‘informative CpG position’. A DNA sequence carrying one or more informative CpG positions in this context is called a ‘marker gene’, regardless whether it is a gene in the common sense or not. For a number of healthy organs and tissues informative CpG sites have been identified (see for example FIG. 5 and FIG. 7) that are specifically methylated. From the pool of different nucleic acids circulating in the bodily fluid, these sites are tested for their methylation status. The specific modifications in these pre-treated nucleic acids caused by said treatment are detected by use of the standard methods as described below.

One preferred embodiment of the method is to perform step two by hybridising specific amplificates of the chemically pretreated DNA with a an oligo array containing oligos specifically detecting said modifications.

Fragments of the chemically pretreated DNA are amplified, using sets of primer oligonucleotides and a, preferably heat-stable, polymerase. The amplification of several DNA segments can be carried out simultaneously in one and the same reaction vessel. Because of statistical and practical considerations, preferably more than two different fragments having a length of 75-2000 base pairs are amplified simultaneously. Usually, the amplification is carried out by means of a polymerase chain reaction (PCR).

The amplificate is performed by means of at least two oligonucleotides wherein one oligonucleotide sequence is reverse complementary and the other identical to an at least 18 base-pair long segment of the chemically pretreated base sequences. Said primer oligonucleotides are preferably characterised in that they do not contain any CpG or TpG dinucleotides. It is one embodiment of the invention that at least one primer oligonucleotide is bound to a solid phase during amplification.

In a particularly preferred embodiment of the method, the sequences of said primer oligonucleotides, and optionally other oligonucleotide probes, are designed so as to selectively anneal to and amplify, only those DNA sequences that are differentially methylated between different tissues or organs, thereby minimising the amplification of background or non relevant DNA. In the context of the present invention, background DNA is taken to mean genomic DNA which does not have a relevant tissue specific methylation pattern, as described in detail in the application WO 02/072880 (as such incorporated by reference).

It is a preferred embodiment that said fragments, obtained by means of the amplification, carry a directly or indirectly detectable label. Preferred are labels in the form of fluorescence labels, radionuclides, or detachable molecule fragments having a typical mass which can be detected in a mass spectrometer, it being preferred that the fragments that are produced have a single positive or negative net charge for better detectability in the mass spectrometer. The detection may be carried out and visualized by means of matrix assisted laser desorption/ionisation mass spectrometry (MALDI) or using electron spray mass spectrometry (ESI).

The amplificates obtained are subsequently hybridised to a set of oligonucleotides and/or PNA (peptide nucleic acid) probes. Preferably this set of probes is arrayed onto a solid phase. The different oligonucleotide sequences can be arranged on a plane solid phase in the form of a rectangular or hexagonal lattice. The solid phase surface is preferably composed of silicon, glass, polystyrene, aluminium, steel, iron, copper, nickel, silver, or gold. However, nitrocellulose as well as plastics such as nylon which can exist in the form of pellets or also as resin matrices may also be used.

In this context, the hybridisation preferably, takes place in the manner described in the following: The set of probes used during the hybridisation is preferably composed of at least 10 oligonucleotides or PNA-oligomers. In the process, the amplificates hybridise to oligonucleotides or PNA-oligomers, which previously bonded to a solid phase. Said oligonucleotides contain at least one base sequence having a length of 10 nucleotides which is reverse complementary or identical to a specific segment of the amplificates' base sequences, the segment containing at least one CpG or TpG dinucleotide. The cytosine of the CpG dinucleotide and respectively the thymidine of the TpG dinucleotide is the 5^{th} to 9^{th} nucleotide from the 5′-end of the 10-mer. One oligonucleotide exists for each CpG or TpG dinucleotide. Said PNA-oligomers contain at least one base sequence having a length of 9 nucleobases which is reverse complementary or identical to a segment of the amplificates' base sequences, the segment containing at least one CpG or TpG dinucleotide. The cytosine of the CpG dinucleotide and respectively the thymidine of the TpG dinucleotide is the 4^{th} to 6^{th} nucleotide seen from the 5′-end of the 9-mer. Preferably one oligonucleotide exists for each CpG or TpG dinucleotide.

It is understood that where it says TpG in this context it can also be replaced by CpA when analyzing the opposite strand, as the amplificates are double stranded DNA. This is obvious to a person skilled in the art and therefore not explicitly mentioned but understood to be equivalent in the scope of the invention.

Next, the non-hybridised amplificates are removed. Finally, the hybridised amplificates are detected. In this context, it is preferred that labels attached to the amplificates are identifiable at each position of the solid phase at which an oligonucleotide sequence or PNA-oligomer is located.

According to the present invention, it is preferred that the labels of the amplificates are fluorescence labels, radionuclides, or detachable molecule fragments having a typical mass which can be detected in a mass spectrometer. The mass spectrometer is preferred for the detection of the amplificates, fragments of the amplificates or of probes which are complementary to the amplificates, it being possible for the detection to be carried out and visualised by means of matrix assisted laser desorption/ionisation mass spectrometry (MALDI) or using electron spray mass spectrometry (ESI). The produced fragments may have a single positive or negative net charge for better detectability in the mass spectrometer.

Another preferred embodiment to perform step two of the invention, that is determining the amount or presence (detectable above a given threshold) of free floating DNA that originates from said tissue, cell type or organ in said sample, is to detect the characteristic modifications in the pre-treated DNA with the use of quantifiable amplification methods such as PCR or isothermal amplification. The selection of suitable primers, probes and reaction conditions can be recognised from that state of the art, but will be described more specifically herein.

It is particularly preferred in that embodiment that the size of the amplified fragment obtained is between 75 and 200 base pairs in length. It is also particularly preferred that said amplificates comprise at least one 20 base pair sequence comprising at least three CpG dinucleotides. Said amplification is carried out using sets of primer oligonucleotides and a preferably heat-stable polymerase. The amplification of several DNA segments can be carried out simultaneously in one and the same reaction vessel. Typically, when the amplification is carried out using a polymerase chain reaction (PCR) the set of primer oligonucleotides includes at least two oligonucleotides, whose sequences are each reverse complementary, identical, or hybridise under stringent or highly stringent conditions to an at least 18-base-pair long segment of the base sequences of suitable marker genes, which are differentially methylated in different tissues, organs or cell types.

In one embodiment of the method, the methylation status of CpG positions within the nucleic acid sequences of said marker genes may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primer pairs contain at least one primer, which hybridises to a bisulfite treated CpG dinucleotide. Therefore, the sequence of said primers comprises at least one CpG, TpG or CpA dinucleotide. MSP primers specific for non-methylated DNA contain a “T′ at the 3′ position of the C position in the CpG. Preferably, therefore, the base sequence of said primers is required to comprise a sequence having a length of at least 18 nucleotides which hybridises to a chemically pretreated nucleic acid sequence of said marker genes and sequences complementary thereto, wherein the base sequence of said oligomers comprises at least one CpG, TpG or CpA dinucleotide. In this embodiment of the method according to the invention it is particularly preferred that the MSP primers comprise between 2 and 4 CpG, TpG or CpA dinucleotides. It is further preferred that said dinucleotides are located near the 3-prime end of the primer, e.g. wherein a primer is 18 bases in length the specified dinucleotides are preferably located within the first 9 bases from the 3-prime end of the molecule. In addition to the CpG, TpG or CpA dinucleotides it is further preferred that said primers should further comprise several bisulfite converted bases (i.e. cytosine converted to thymine, or on the hybridising strand, guanine converted to adenine). In a further preferred embodiment said primers are designed so as to comprise no more than 2 cytosine or guanine bases.

The fragments obtained by means of the amplification can carry a directly or indirectly detectable label. Preferred are labels in the form of fluorescence labels, radionuclides, or detachable molecule fragments having a typical mass which can be detected in a mass spectrometer. Where said labels are mass labels, it is preferred that the labelled amplificates have a single positive or negative net charge, allowing for better detectability in the mass spectrometer. The detection may be carried out and visualised by means of, e.g., matrix assisted laser desorption/ionisation mass spectrometry (MALDI) or using electron spray mass spectrometry (ESI).

In a particularly preferred embodiment of the method the amplification is carried out in the presence of at least one species of blocker oligonucleotides. The use of such blocker oligonucleotides has been described by Yu et al., BioTechniques 23:714-720, 1997. The use of blocking oligonucleotides enables the improved specificity of the amplification of a subpopulation of nucleic acids. Blocking probes hybridised to a nucleic acid suppress, or hinder the polymerase mediated amplification of said nucleic acid. In one embodiment of the method blocking oligonucleotides are designed so as to hybridise to background DNA, that is DNA that is not tissue, cell type or organ specific methylated. In a further embodiment of the method said oligonucleotides are designed so as to hinder or suppress the amplification of unmethylated nucleic acids as opposed to methylated nucleic acids or vice versa.

Blocking probe oligonucleotides are hybridised to the bisulfite treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridise to the bisulfite treated nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a ‘TpG’ at the position in question, as opposed to a ‘CpG.’

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivatised at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-terminii thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridised blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase.

In one embodiment of the method, the binding site of the blocking oligonucleotide is identical to, or overlaps with that of the primer and thereby hinders the hybridisation of the primer to its binding site. In a further preferred embodiment of the method, two or more such blocking oligonucleotides are used. In a particularly preferred embodiment, the hybridisation of one of the blocking oligonucleotides hinders the hybridisation of a forward primer, and the hybridisation of another of the probe (blocker) oligonucleotides hinders the hybridisation of a reverse primer that binds to the amplificate product of said forward primer.

In an alternative embodiment of the method, the blocking oligonucleotide hybridises to a location between the reverse and forward primer positions of the treated background DNA, thereby hindering the elongation of the primer oligonucleotides.

It is particularly preferred that the blocking oligonucleotides are present in at least 5 times the concentration of the primers.

The amplificates obtained are analysed in order to ascertain the methylation status of the informative CpG dinucleotides prior to the treatment.

In embodiments where the amplificates are obtained by means of MSP amplification and/or blocking oligonucleotides, the presence or absence of an amplificate is in itself indicative of the methylation state of the CpG positions covered by the primers and or blocking oligonucleotide, according to the base sequences thereof. All possible known molecular biological methods may be used for this detection, including, but not limited to gel electrophoresis, sequencing, liquid chromatography, hybridisations, real time PCR analysis or combinations thereof. This step of the method further acts as a qualitative control of the preceding steps.

Amplificates obtained by means of both, standard and methylation specific PCR, are further analysed in order to determine the CpG methylation status of the free floating DNA in said sample. This may be carried out by means of hybridisation-based methods such as, but not limited to, array technology and probe based technologies as well as by means of techniques such as sequencing and template directed extension.

In yet a further embodiment of the method, the genomic methylation status of the informative CpG positions may be ascertained by means of oligonucleotide probes that are hybridised to the bisulfite treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; see also U.S. Pat. No. 6,331,393). There are two preferred embodiments of utilising this method. One embodiment, known as the TaqMan™ assay employs a dual-labelled fluorescent oligonucleotide probe. The TaqMan™ PCR reaction employs the use of a nonextendible interrogating oligonucleotide, called a TaqMan™ probe, which is designed to hybridise to a CpG-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. Hybridised probes are displaced and broken down by the polymerase of the amplification reaction thereby leading to an increase in fluorescence. For analysis of methylation within nucleic acids subsequent to bisulfite treatment, it is required that the probe be methylation specific, as described in U.S. Pat. No. 6,331,393, (hereby incorporated by reference in its entirety) also known as the MethylLight™ assay. The second preferred embodiment of this technology is the use of dual-probe technology (Lightcycler™), each probe carrying donor or recipient fluorescent moieties. The hybridisation of the two probes in proximity to each other is indicated by an increase or decrease in fluorescence. Both these techniques may be adapted in a manner suitable for use with bisulfite treated DNA, and moreover for methylation analysis within CpG dinucleotides.

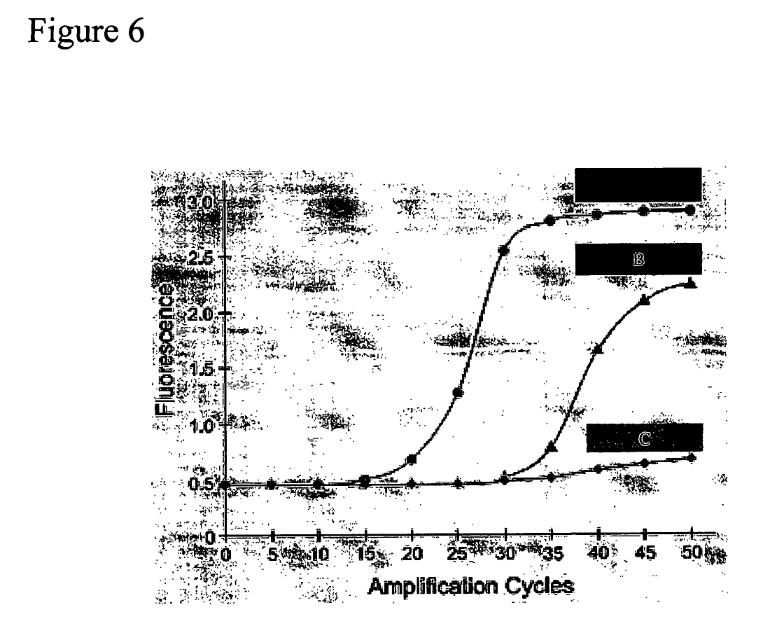

Quantification of said methylation determination assays can easily be done by introducing an internal standard DNA of known quantity and known methylation status, as it is routinely done in the art (see FIG. 6 for illustration and Nakao et al. (2000) Cancer Research 60: 3281-9.)

In yet a further embodiment of the method, the second step of the method, that is identifying the tissue, organ or cell type that significantly contributes to the free floating DNA, comprises the use of template-directed oligonucleotide extension, such as MS-SNuPE as described by Gonzalgo & Jones, Nucleic Acids Res. 25:2529-2531, 1997.

In yet a further embodiment of the method, the second step of the method, that is identifying the tissue, organ or cell type that significantly contributes to the free floating DNA, comprises sequencing and subsequent sequence analysis of the amplificates generated with a method described above (Sanger F et al. (1977) Proc Natl Acad Sci USA 74: 5463-5467).

The methylation patterns found in the tested sample will be identified as belonging to a certain tissue, cell type or organ.

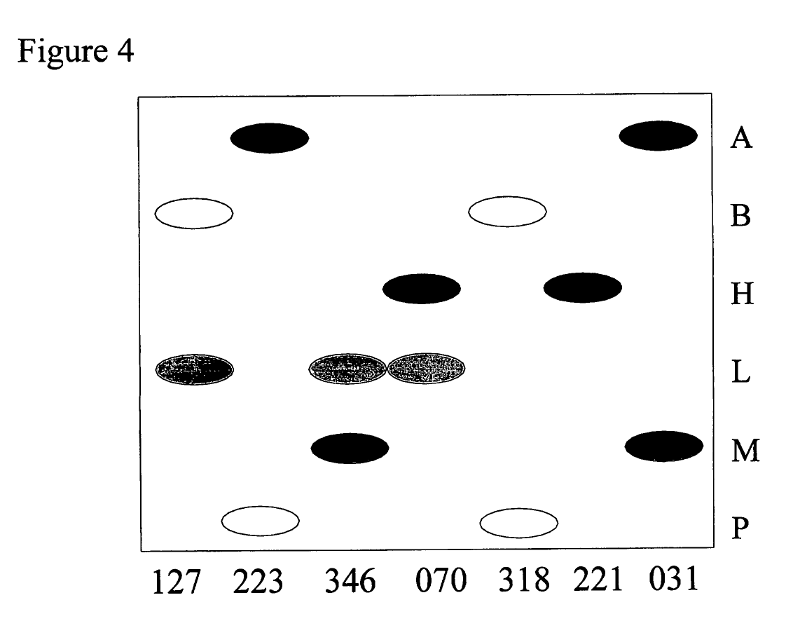

This is done either by comparing the individual data set resulting from said analysis to data received in previous studies or to a dataset obtained in a parallel experiment on one or preferably more control fluids. The data received in previous studies will comprise of typical methylation patterns of either a single marker gene or a set of marker genes determined in different DNA samples derived from different organs, cell types or tissues. These characteristic differences in said DNA methylation patterns, that can be correlated to the source of tissue, organ or cell type said DNA derived from are identified and stored as a valuable dataset. In FIG. 4 a schematic drawing is presented to visualise said principle.

If a CpG positions is only ever specifically methylated when the corresponding DNA sequence was isolated from kidney cells but said CpG position is not methylated when the DNA was isolated from a liver cell, a blood cell, a bladder cell or colon cell etc. said CpG position is an informative CpG position. A gene carrying one or more informative CpG positions is called a marker gene. The more comparative studies have been made on the methylation statuses of said positions in correlation with its tissue of origin the higher is the quality of the corresponding marker gene. The most reliable information on the DNA's origin will be extracted from the analysis of several of those marker genes simultaneously, by employing a panel of such marker genes.

This analysis will reveal if a significant part of the free floating DNA analysed can be identified as belonging to a specific tissue, organ or cell type.

In the third step of the method, it is concluded whether a medical condition such as cell proliferative or inflammatory disease at the specified source is causing the release of DNA into the bodily fluid. The presence or absence of a medical condition in said organ is determined by comparing the individual's test result with the dataset that was built up in house in previous studies. Wherein the extracellular DNA can clearly be correlated to a specific organ or tissue as the predominant source a further analysis of said organ or tissue—or a further analysis of said DNA by means of cancer marker genes—as described elsewhere—is highly indicated.

In a preferred embodiment additional optional steps are added to the method according to this invention.

In said preferred embodiment, the first result of an analysis of a bodily fluid from a screen would be an information about the level of circulating DNA. In cases where this is elevated above normal (average from healthy people), which so far has not been seen as a significant risk factor on its own, would now lead to a further analysis in terms of methylation analysis. Without having to guess, which kind of organ might be affected, and as such might be responsible for the emission of those DNA levels and without needing to employ assays on certain tumour marker genes, with this invention it will be possible to reveal the DNA's origin. This is based on the detection of tissue specific methylation patterns on pre-selected tissue marker genes. Those genes contain informative CpG positions, CpG positions that are differentially methylated, specifically for the tissue the DNA has been isolated from. Such marker genes have been described by Adorjan et al. (2002, Nucleic Acids Res. 30, e21). With the use of tissue-, organ- or cell type-specific methylation marker genes it is possible to interpret a specific methylation pattern as belonging to a specific tissue type.

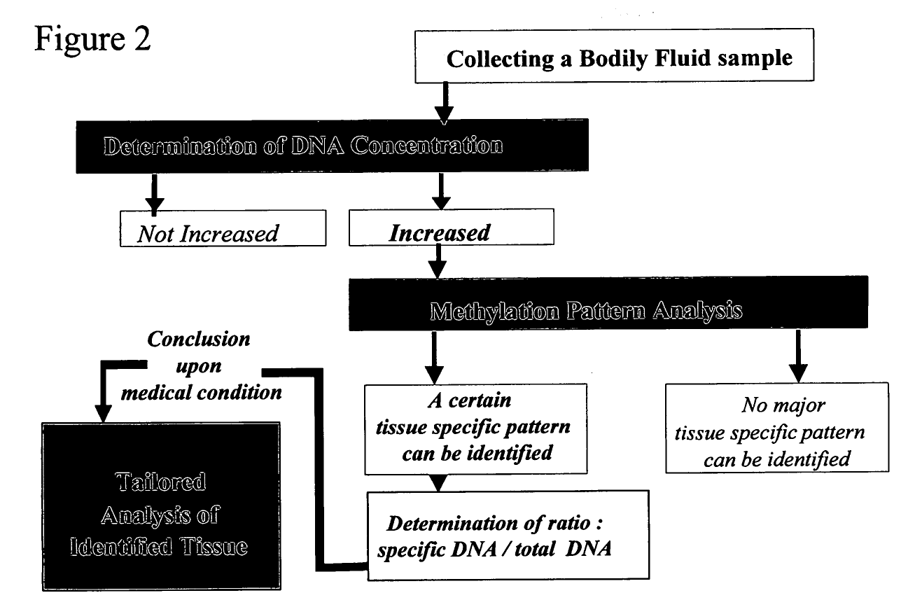

In FIG. 2 a flow chart gives an overview of said embodiment. The first optional step added to the described method is the determination of the total amount of free floating DNA prior to determination of the amount of free floating DNA that originates from a specific tissue. In said optional additional step, prior to step 2, the free floating DNA in said bodily fluid is quantified as it is described now:

The quantitation of the total amount free floating DNA may be done by any means that are standard to one skilled in the art. Commonly used techniques are based on spectrophotometric and/or fluorometric analyses, for example: the concentration of a dilute sample of plasmid DNA purified by two passes through an ethidium bromide—caesium chloride (EtBr—CsCl) centrifugation gradient can either be determined on an for example LKB Biochrom Ultrospec II spectrophotometer for absorbance at wavelengths of 260 nm and 280 nm, or it can be tested for emission of 460 nm on the Hoefer TKO 100 mini-fluorometer in the presence of bisbenzimidizole, a fluorescent dye known as Hoechst H 33258 (manufactured by American Hoechst Corporation), that has an excitation maximum at 356 mm and an emission maximum of 458 when bound to DNA. The spectrophotometer detects absorbance due to RNA as well as DNA, while the Hoechst dye used in the fluorometer interacts specifically with adenosine and thymidine residues of DNA. In a preferred embodiment the Invitrogen's nucleic acid quantitation DNA Dipstick™ kit is used, which is claimed to be sensitive enough to detect as little as 0.1 ng/ul of nucleic acid. Unfortunately, the method cannot be used with samples containing more than 10 ng/ul of nucleic acids (Hengen P N (1994) Trends in Biochemical Sciences 19,93-94 and discussion thereof pp 46-47).

It is preferred that the total amount of free floating DNA is measured by intercalating fluorescent dyes or other dyes changing their fluorescence properties when binding to DNA, and also by hybridisation to DNA specific probes including, but not limited to oligonucleotides or PNA (peptide nucleic acid) oligomers, real time PCR assays or other real time amplification procedures, UV-Vis absorbance or in general amplification procedures with subsequent determination of the amount of product formed.

Wherein said optional step has been performed, it is also preferred that in another additional fourth step, the presence or absence of a medical condition in said organ is determined by comparing the individual's test result, regarding the fraction of free floating DNA that originates from a specific source with the dataset that was built up in house in previous studies. Said fraction is determined by building the ratio of the amount of free floating DNA that can be correlated to a specific cell type, tissue or organ as source, and the amount of total free floating DNA. Based on these results it is possible to identify patients with abnormal amounts of DNA of a certain organ or tissue, as in increased by more than 10% above a value defined as “normal”, in their bodily fluids. In a preferred embodiment it is possible to positively identify patients with free floating DNA levels increased by at least but not limited to 20% above a value defined as normal. In a further preferred embodiment it is possible to identify patients with an increased level of free floating DNA, specified in increased by at least but not limited to 40% above normal.

Furthermore and most importantly said analysis will not only tell that the patients DNA level are increased but also reveal the possible cause of it, as in specifying where this extracellular DNA comes from. This will give the physician or clinician involved a valuable tool to identify a disease in its very early days, even before noticeable symptoms might have occurred.

The invention provides a method as described above characterised in that said methylation pattern is found to be specific for said organ, cell type or tissue with regards to other organs, cell types or tissues. For example, a specific CpG methylation pattern occurs only when the DNA analysed originates from colon cells, but not when the DNA analysed originates from any other cell.

In a preferred embodiment the method is characterised in that said methylation pattern is found to be specific for said organ or tissue with regards to methylation patterns that can be found in DNA from other organs or tissues, specified by the fact that it is not found in other organs or tissues which are involved in the medical condition of interest and thereby independent of the medical condition the patient might be diagnosed with.

For example, a CpG position may be methylated when the DNA originates from an inflamed cell in kidneys, but it could be not methylated in other inflamed cells around and close by the kidney, however said CpG position might be methylated in cancerous lung cells.

In a further preferred embodiment the method is characterised in that said methylation pattern is found to be specific for said organ or tissue with regards to other organs or tissues when the medical condition the patient is diagnosed with is a tumour or another cell proliferative disease.

The invention provides a method for detecting the absence or presence of a medical condition in an organ, tissue or cell type characterised in that the following steps are carried out: First, retrieving a bodily fluid sample from an individual as described above; second, determining the amount or presence (detectable above a given threshold) of free floating DNA that has a tissue, cell type or organ specific DNA methylation pattern; third, concluding whether an abnormal level of free floating DNA that originates from said tissue, cell type or organ is present. In a preferred embodiment in an additional fourth step it is concluded whether a medical condition associated with said tissue, cell type or organ is present.

It is a preferred embodiment of the invention wherein said method for detecting the absence or presence of a medical condition in an organ, cell type or tissue, is characterised in that more optional steps are carried out: First, retrieving a bodily fluid sample from an individual as described above; second, detecting the amount of total free floating DNA in said sample as described above; third determining the amount of free floating DNA that originates from a specific tissue cell type or organ by determining the amount of free floating DNA that has a DNA methylation pattern characteristic for said tissue, cell type or organ; fourth, determining the fraction of said free floating DNA which originates from said specific tissue, cell type or organ out of the total free floating DNA; fifth, concluding, whether there is an abnormal level of total free floating DNA and whether a significant part of the total free floating DNA originates from said tissue, cell type or organ and sixth, concluding whether a medical condition associated with said tissue or organ is present.

In a further embodiment the invention provides a method for determining the fraction of free floating DNA in a bodily fluid that originates from an organ, cell type or tissue of interest, characterised in that the following steps are carried out: First, retrieving a bodily fluid sample from an individual; second, conditioning said sample to prepare the binding of free floating DNA to a surface; third, detecting the amount of total free floating DNA by measuring the amount of DNA bound to said surface; fourth, subjecting said surface comprising said immobilised DNA to a chemical and/or enzymatic treatment that converts all unmethylated cytosines in the DNA into uracil but leaving in position 5 methylated cytosines unchanged as described above; fifth, amplifying the treated DNA; sixth, analysing several positions in said treated DNA and determining the amount or presence (detectable above a given threshold) of DNA that has a tissue, organ or cell type specific DNA methylation pattern; seventh, determining the fraction of free floating DNA that originates from said tissue or organ out of the total free floating DNA.

In a further embodiment the method as described above includes the following additional steps: If there is an abnormal level of total free floating DNA it is concluded whether this DNA originates from said tissue or organ and whether a medical condition associated with said tissue or organ is present.

The present invention is also directed to a method for diagnosing a disease or medical condition that comprises any of the methods that are disclosed in this invention.

It is preferred that the method that is subject to the present invention is used for diagnosing a disease or medical condition. It is also preferred that said method is used to guide a physician's or practitioner's selection on employing further diagnostic tests.

In addition the invention discloses the means to produce a device to determine the total amount of free floating DNA in a bodily fluid, comprising a surface to bind DNA floating in a sample volume of bodily fluid and a means for detecting the amount of DNA bound to this solid surface. The device is further characterised in that it comprises a chamber to host the surface and reagents to chemically or enzymatically modify the DNA bound to said surface and a means to control and adjust the temperature in this chamber.

Said surface may be the same as described and used in the DNA DipStick™ kit (supplied by Invitrogen) or of other means enabling DNA to selectively bind to a material applicated to some unspecified kind of carrier, which might be either mobile or fixed. The binding may for example be based on unspecific hybridisation of nucleic acids. The quantification of DNA bound to said surface may be carried out by any means standard to anyone skilled in the art or for example following instructions given in the DNA DipStick™ Kit. Furthermore the invention discloses the means how to produce a chamber or similar kind of closed environment to host said surface together with the required reagents and/or enzymes to modify the DNA bound to said solid surface.

The means to control and adjust the temperature in this chamber may be done by means that are standard to anyone skilled in the art, for example by fixing an electronic thermometer or any device able to read the temperature and connect it to a chip programmed to react in a certain way by switching on a cooling or heating unit.

However, a kit along the lines of the present invention can also contain only parts of the aforementioned components and may not include the device. It may be composed, for example, of a bisulfite-containing reagent, a set of primer oligonucleotides containing at least two oligonucleotides whose sequences in each case correspond or are complementary to a 18 base long segment of a specific base sequence, oligonucleotides and/or PNA (peptide nucleic acid)-oligomers as well as instructions for carrying out and evaluating the described method.

The idea to combine the analysis of free floating DNA in a biological sample like blood with the consequent analysis of its origin by means of methylation state analysis leads to new possibilities of screening large populations for very early signs of, for example, cancer even before clinical stages, when no other symptoms are noticeable yet and no hints are available to the patient or physician as to what to look for. As early detection is the most important step in fighting a disease like cancer this method provides an important improvement towards a successful fight against these diseases. In addition the method can, for example, be employed to monitor the progression of a tumour (metastasis) after treatment and thereby allows to optimise the dosage of said treatment or adjusting to a different treatment in a patient specific individual manner.
SEQ ID No. 1 shows a first primer for beta actin: TGGTGATGGAGGAGGTTTAGTAAGT;
SEQ ID No. 2 shows a second primer for beta actin: AACCAATAAAACCTACTCCTCCCTTAA; and
SEQ ID No. 3 shows a probe for beta actin: ACCACCACCCAACACACAATAACAAACCA.
SEQ ID No. 4 shows a forward Primer: GGTGATTGTTTATTGTTATGGTTTG for the EYA4 gene;
SEQ ID No. 5 shows a reverse Primer: CCCCTCAACCTAAAAACTACAAC for the EYA4 gene;
SEQ ID No. 6 shows a forward Blocker: GTTATGGTTTGTGATTTTGTGTGGG for the EYA4 gene;
SEQ ID No. 7 shows a reverse Blocker: AAACTACAACCACTCAAATCAACCCA for the EYA4 gene; and
SEQ ID No. 8 shows a probe: AAAATTACGACGACGCCACCCGAAA for the EYA4 gene.

FIG. 1:

FIG. 1 shows a flow chart that gives an overview of the method that is subject of the invention as described.

FIG. 2:

FIG. 2 shows a flow chart that gives an overview of the method including optional steps that is described as the preferred embodiment.

FIG. 3:

FIG. 3 shows the results of an experiment wherein the levels of free floating DNA in serum samples have been determined in relation to the presence and absence of a disease. The DNA was extracted using a Qiagen UltraSens kit, and quantified with a picogreen fluorescence assay. The values shown at the Y-axis are given in nanograms per millilitre. The different columns relate to the sample sources: DNA levels in serum samples from healthy donors (column B) have been compared with DNA levels in serum samples of 18 lung cancer patients (column C), 19 colon cancer patients (column D) and 24 breast cancer patients (column E). (Column A gives a value for the level of DNA in plasma samples of healthy donors.) The levels of free DNA in serum from each of the diseased groups (columns C, D and E) is around 200 nanograms per millilitre or higher. This, firstly, confirms that average levels in serum samples from cancer patients are significantly higher than the average levels of DNA in serum samples from healthy donors, and secondly it shows that there is a sufficient amount of DNA for the analysis of methylation patterns.

FIG. 4:

FIG. 4 is a schematic image showing how different methylation patterns can be correlated to different organs. Circles indicate a methylated CpG position. The different numbers indicate different informative CpG positions within the genome, which show organ specific methylation patterns. When a circle is missing at the same column in a different line that same CpG is not methylated. The letters at the right side indicate different organs as follows: A: Adipose; B: Breast; H: Liver; L: Lung; M: Muscle and P: Prostate.

FIG. 5:

In FIG. 5 the result of a study is shown, wherein the DNA methylation pattern of specific CpGs (1-10) in DNA from kidney detected on specific marker DNA has been compared with the DNA methylation pattern detected on the same marker DNA when said DNA originates from prostate. The letters above the image indicate whether the sample is a kidney sample (Z) or a prostate sample (Y). The letters below the image indicate the different samples that have been analysed. 20 different prostate samples (A-S) and 18 different kidney samples (A-R) were analysed. Specific CpG positions that were expected to be differentially methylated were analysed as to their capacity of differentiating those tissues. The isolated and bisulfite treated DNA was amplified and labelled according to its source of tissue. Said amplificates were hybridised with a set of oligos arrayed on a solid surface (Adorjan et al. (2002) Nucleic Acids Res. 30, e21). Said oligos were designed as to hybridise against those specific CpG containing sequences only if they were methylated prior to treatment or only if they were not methylated prior to treatment. The numbers at the right side of the figure indicate the different CpG positions, some of which belong to the same gene. From which genes the tested CpGs can be correlated is given in the following list:

The informative CpG positions were found either in the genes or their regulatory regions:
1: APOC 2
2: WT 1 (Wilm's Tumor gene)
3: DAD 1
4: c-myc
5: UBB
6: ATP6
7-10: GP1 BB (four different CpG positions)

For further information see Adorjan an et al. (2002) Nucleic Acids Res. 30, e21.

FIG. 6:

FIG. 6 shows how a specific DNA can be quantified by using hybridisation probes in a real-time PCR method. Wherein the method requires hybridisation of both labelled oligonucleotides to defined sequences within the amplification product, as in the lightcycler technology, fluorescence is generated, indicating the amplification of said specific fragment. It is then determined, how many cycles it takes until the signal increases dramatically. This is designated as the so called “threshold cycle number”. By comparing said number with the threshold cycle numbers of standard samples of known DNA quantity, the template quantity can easily be determined. In FIG. 6 at the x-axis the number of amplification cycles is indicated. At the y-axis the level of fluorescence is given. Curve A is the lightcycler result for a template of a concentration of 104 copies, curve B is for a concentration of 10 copies and curve C shows the result for a template that is not present at all (0 copies). Even at very low template concentrations practically no unspecific signal can be observed even after more than 30 cycles. Thus, DNA-quantification with hybridisation probes is not only sensitive but also highly specific.

FIG. 7:

In FIG. 7 the result of a study is shown, wherein the DNA methylation pattern of specific CpGs (1-10) in DNA from four different tissues has been analysed. The letters above the image indicate whether the samples that were analysed were derived from brain tissue (R, 4 samples), breast tissue (B, six samples), liver tissue (H, two samples) or muscle tissue (M, five samples). FIG. 7 shows how specific informative CpG positions within the genome are specifically methylated according to the organ, tissue or cell type the analysed DNA is derived from. Each row shows the specific methylation analysis result for one CpG position. These data have been obtained by bisulfite sequencing and were translated into this image with a visualisation tool, wherein a high degree of methylation at that CpG position is indicated by the colour at the top of the coloured strip provided at the left of the picture, and a very low degree of methylation is indicated by the colour at the bottom of said coloured strip.

### EXAMPLE 1

Organ Specific Methylation Pattern Analysis on Plasma Samples

A blood sample was taken from a patient who was unaware that he had been exposed to high levels of radiation during his years of service at the army. Now he wishes to know whether he has developed a neoplastic disease like a tumour. His physician has not yet found any typical symptoms other than the patient complaining about unspecific pain at different organs, including headache.

A 20 ml blood sample was collected in heparin. Plasma and lymphocytes were separated by Ficoll gradient. Control lymphocyte and plasma DNA were purified on Qiagen columns (Qiamp Blood Kit, Qiagen, Basel, Switzerland) according to the “blood and body fluid protocol”. Plasma was passed on the same column. After purification of about 10 ml of plasma 350 ng of DNA were obtained. The DNA was subjected to a sodium bisulfite treatment as it has been described in Olek A, Oswald J, Walter J. (1996) A modified and improved method for bisulphite based cytosine methylation analysis. Nucleic Acids Res. 24: 5064-6. An aliquot of this bisulfite treated DNA was used for methylation analysis based on sequencing. The individual's test result was compared with the dataset obtained from previous samples of known tissues and cell types as it is shown in FIG. 7. From that it could be concluded that a significant portion of the DNA in the patient's blood was derived from his lung. Said result was send back to the physician who now referred the patient to a hospital that is specialised on inflammatory or cell proliferative diseases of the lung.

### EXAMPLE 2

Organ Specific Methylation Pattern Analysis on Serum Samples

A blood sample was taken from a patient, who wishes to know whether he has developed a neoplastic disease like a tumour. His physician has not yet found any typical symptoms other than the patient complaining about randomly occurring unspecific pain in his stomach, recurrent headache and pain in his kidneys.

A serum sample has been taken from the patient. DNA has been isolated from the serum with the use of the Qiamp kit and has been bisulfite treated as described in Example 1.

A typical methylation pattern could be determined analysing the methylation statuses of a higher number of different informative CpG sites, that were used as markers for different tissues and organs, simultaneously. That was done by first amplifying the relevant fragments with the use of specific primers designed as to only specifically amplify those fragments of the bisulfite treated DNA that contain informative CpG positions. These amplificates were labelled with a fluorescent dye. A set of detection oligos, each designed as to specifically only hybridise with the amplified version of the bisulfite treated nucleic acid that was methylated as it is characteristic for a specific organ. The detection oligos contain a CG when said CpG position is methylated in a specific organ or tissue (or a TG where said CpG position is unmethylated in a specific organ or tissue). These oligos were fixed to a solid surface as to provide a chip. The labelled amplificates were hybridised with said chip and non hybridising amplificates were removed. The signal pattern on the chip was then translated in a methylation pattern, indicative of a specific organ.

The analysis of the patient's DNA methylation patterns, led to the conclusion that a significant portion of the DNA originated from colon.

The physician therefore initiated a second analysis on said bisulfite treated DNA. He required the patient's DNA to be tested a second time, this time specifically only with the colon marker EYA 4. A predominant signal could be detected using the following EYA4-HeavyMethyl MethyLight assay. The methylation status was determined with a HM MethyLight assay designed for the CpG island of the EYA4 colon marker gene and a control gene was assayed in parallel. The CpG island assay covers CpG sites in both the blocking oligos and the taqman® style probe, while the control gene does not.

Methods. The CpG island assay (methylation assay) was performed using the following primers and probes:

Control gene: beta actin (Eads et al., 2001):

| | |
|---|---|
| Primer: | |
| TGGTGATGGAGGAGGTTTAGTAAGT; | (SEQ ID No. 1); |
| Primer: | |
| AACCAATAAAACCTACTCCTCCCTTAA; | (SEQ ID No. 2) |
| and | |
| Probe: | |
| ACCACCACCCAACACACAATAACAAACCA | (SEQ ID No.3) |
| Forward Primer: | |
| GGTGATTGTTTATTGTTATGGTTTG | (SEQ ID No.4) |
| Reverse Primer: | |
| CCCCTCAACCTAAAAACTACAAC | (SEQ ID No.5) |
| Forward Blocker: | |
| GTTATGGTTTGTGATTTTGTGTGGG | (SEQ ID No.6) |
| Reverse Blocker: | |
| AAACTACAACCACTCAAATCAACCCA | (SEQ ID No.7) |
| Probe: | |
| AAAATTACGACGACGCCACCCGAAA. | (SEQ ID No. 8) |

The reactions were each run in triplicate on the individual's DNA sample with the following assay conditions:
Reaction solution: (400 nM primers; 400 nM probe; 10 μM both blockers; 3.5 mM magnesium chloride; 1×ABI Taqman buffer; 1 unit of ABI TaqGold polymerase; 200 μMdNTPs; and 7μl of a solution containing 50 ng of DNA, in a final reaction volume of 20 μl);
Cycling conditions: (95° C. for 10 minutes); (95° C. for 15 seconds, 64° C. for 1 minute (2 cycles)); (95° C. for 15 seconds, 62° C. for 1 minute (2 cycles); (95° C. for 15 seconds, 60° C. for 1 minute (2 cycles)); and (95° C. for 15 seconds, 58° C. for 1 minute, 60° C. for 40 seconds (41 cycles)).

The amplification of said fragment indicated the presence of a specific methylation pattern in said informative CpG positions (of EYA 4). From comparing the test result and the intensity of the fluorescent signal with a data set obtained from other samples it could be concluded that a significant part of the DNA in the patients sample originated from colon. This result allowed the physician to refer said patient to an expert in gastrointestinal diseases.

### EXAMPLE 3

In another case the physician was following a different strategy. He was first testing for the total amount of free floating DNA in said patient's serum, because this test is less cost intense and was covered by the patient's insurance. The blood sample was sent to a laboratory. After having separated the plasma from blood cells by centrifugation at 3000 g for 20 min the DNA from the blood plasma was extracted using the QIAamp Blood Kit (Qiagen, Hilden, Germany) using the blood and body fluid protocol referring to Wong et al. (1999), Cancer Res 59: 71-73 and Lo et al. (1998) Am. J. Genet. 62: 768-775. It was determined that the level of total free floating nucleic acids in said serum sample was 20 times higher than it usually is in samples from healthy donors, that are not suffering from cell proliferative diseases. The data that were establishing this “normal” value have been obtained in previous studies based on a high number of samples and were approved by the regulatory agencies. These data had been stored in their dataset.

Knowing that his patient had a level of free floating DNA in his serum that was 20 times higher than the average allowed the physician to diagnose a high likelihood of his patient to suffer from a cell proliferative disease. With this diagnosis the insurance was willing to pay for a more informative test as to further specify the kind of disease.

The physician now requested the methylation analysis of said DNA with the aim to determine where the free floating DNA in the serum of his patient originated from. Said DNA was treated with sodium bisulfite as described above. The methylation pattern analysis was carried out with the use of a number of informative CpG site containing marker nucleic acids and the collected datasets from other samples to compare the results with (as illustrated in FIG. 4). Said analysis revealed that a significant portion of said free floating DNA originated from liver. At this point the physician referred the patient to an oncologist specified for liver tumours.

### EXAMPLE 4

A research team is interested in identifying risks of developing lung specific diseases like for example lung cancer in a population, that has been exposed to specific environmental conditions.

As these conditions only developed during the recent years no data are available on an accumulated occurrence of cancer in said population yet. Therefore they are employing an analysis of said individuals bodily fluids as to whether they can find early signs of developing diseases. Sputum samples have been collected from a high number of individuals.

Those sputum samples were analysed as follows: Sputum samples were spun at 3000×g for 5 min and washed twice with phosphate-buffered saline. Cell pellets were digested with 1% SDS/proteinase K, and DNA was extracted and purified using Qiagen columns (Qiamp Blood Kit, Qiagen, Basel, Switzerland) according to the “blood and body fluid protocol”. The DNA obtained was subjected to a sodium bisulfite treatment as it has been described in Olek A, Oswald J, Walter J. (1996) A modified and improved method for bisulphite based cytosine methylation analysis. Nucleic Acids Res. 24: 5064-6. An aliquot of this bisulfite treated DNA was used for methylation analysis. As the study was designed to only look for lung diseases, the analysis was restricted to the use of informative CpG sites that are specifically methylated in lung cells, but unmethylated in other cells that might potentially occur in a sputum sample. The methylation analysis was based on sensitive detection assays. First results were obtained with the use of a HM assay, as it is described in here. Primers were designed to amplify a fragment that contains seven different CpG sites that are all methylated only in DNA from lung cells. Blocking oligos were designed that hybridised to two of those sites in the bisulfite treated DNA, only when said CpG sites were unmethylated prior to bisulfite treatment. A pair of Lightcycler probes was designed as to only bind to the amplified fragment of the bisulfite treated DNA when two different informative CpG sites were methylated. That way the presence was indicated by the generation of a fluorescent signal and the amount of said lung derived DNA in the total amount of DNA was quantified by the number of cycles required to generate a detectable signal in comparison to signals generated by standardised amounts of control DNA.

The primary test results have been confirmed with the use of MSP primers in combination with the use of Taqman probes. MSP primers were designed to specifically bind to the bisulfite treated sequence containing two and three of those CpG sites that were methylated in lung cells, but not in other cells. The Taqman probe was designed to bind to the other two CpG sites in said amplified product only when those were unmodified after treatment with bisulfite (methylated cytosines prior to treatment). Therefore the presence of an amplification product, indicated by the fluorescent signal of the Taqman probe confirmed the primary results.

As the majority of the individuals did not show free floating DNA in their sputum samples that exhibited methylation pattern characteristic for lung, it was concluded that they did not contain lung derived DNA in their sputum samples. It was concluded that said population did not develop lung specific cell proliferative diseases and as such there was no reason to believe that said environmental conditions were adding to the risk of developing a neoplastic or inflammatory diseases like lung cancer or lung inflammation.

1. A method for detecting the presence or absence of a medical condition in a tissue, cell type or organ of an individual, comprising the following steps:
a) retrieving a bodily fluid sample from said individual;
b) determining the amount or presence of free floating DNA that originates from said tissue, cell type or organ in said sample; and
c) determining the presence or absence of a medical condition based on the amount or presence of free floating DNA that originates from said tissue, cell type or organ.

2. A method for detecting the presence or absence of a medical condition in a tissue, cell type or organ of an individual, comprising the following steps:
a) retrieving a bodily fluid sample from said individual;
b) determining the amount of total free floating DNA in said sample;
c) determining the amount of free floating DNA that originates from said tissue, cell type or organ in said sample; and
d) determining the presence or absence of a medical condition based on the total amount of free floating DNA and the fraction of free floating DNA that originates from said tissue, cell type or organ.

3. The method according to claim 1 or 2, characterised in that the sample is conditioned before the amount or presence of free floating DNA is determined.

4. The method according to claim 3, characterised in that the sample is conditioned by means of centrifugation, filtering, heating, cooling, concentration or chemical treatment.

5. The method according to one of the preceding claims, characterised in that the amount or presence of DNA originating from a certain organ or tissue is determined by analysing a DNA methylation pattern that is characteristic for said organ, tissue or cell type.

6. The method according to claim 5, characterised in that said methylation pattern is characteristic for said organ, tissue or cell type and not found in other organs, tissues or cell types involved in the medical condition of interest.

7. The method according to any of the preceding claims, characterised in that the medical condition is a cell proliferative and/or neoplastic disease.

8. The method according to any of the preceding claims, characterised in that the samples are obtained from bodily fluids like whole blood, blood plasma, blood serun, urine, sputum, ejaculate, semen, tears, sweat, saliva, lymph fluid, bronchial lavage, pleural effusion, peritoneal fluid, meningal fluid, amniotic fluid, glandular fluid, fine needle aspirates, nipple aspirate fluid, spinal fluid, conjunctival fluid, vaginal fluid, duodenal juice, pancreatic juice, bile and cerebrospinal fluid from said individual.

9. The method according to one of the preceding claims, characterised in that the methylation pattern is determined by subjecting the DNA to a chemical or enzymatic treatment that converts all unmethylated cytosines in the DNA into uracil but leaves position 5-methylated cytosines unmodified.

10. A method for detecting the absence or presence of a medical condition in an organ, cell type or tissue, comprising performing the following steps:
a) retrieving a bodily fluid sample;
b) determining the amount or presence of free floating DNA that exhibits a tissue-, organ- or cell type-characteristic DNA methylation pattern;
c) concluding, whether there is an abnormal level of free floating DNA that originates from said tissue, cell type or organ; and
d) concluding, whether a medical condition associated with said tissue, cell type or organ is absent or present.

11. A method for detecting the absence or presence of a medical condition in a specific organ, cell type or tissue, comprising the following steps:
a) retrieving a bodily fluid sample;
b) detecting the amount of total free floating DNA in said sample;
c) determining the amount of free floating DNA that originates from said specific tissue, cell type or organ by determining free floating DNA that exhibits a tissue-, cell type- or organ-characteristic DNA methylation pattern;
d) determining the fraction of total free floating DNA that originates from said specific tissue, cell type or organ;
e) concluding, whether an abnormal level of free floating DNA that originates from said specific tissue, cell type or organ is present; and
f) concluding, whether a medical condition associated with said specific tissue, cell type or organ is absent or present.

12. A method for determining the fraction of free floating DNA in a bodily fluid that originates from an organ, cell type or tissue of interest, comprising the following steps:
a) retrieving a bodily fluid sample;
b) conditioning said sample in order to allow a binding of free floating DNA to a surface;
c) binding an essential fraction of said total free floating DNA to said surface;
d) detecting the amount of total free floating DNA by measuring the amount of DNA bound to said surface;
e) subjecting said surface comprising said bound DNA to a chemical and/or enzymatic treatment that converts all unmethylated cytosines in the DNA into uracil but leaves position-5 methylated cytosines unmodified;
f) amplifying the treated DNA;
g) analysing several methylation-specific positions in said treated DNA, and thereby determining the amount of DNA that exhibits a tissue, cell type or organ-characteristic DNA methylation pattern; and
h) determining the fraction of total free floating DNA that originates from said tissue, cell type or organ.

13. The method of claim 12, comprising the following additional steps:
i) concluding, whether said DNA originates from said tissue, cell type or organ, if there is an abnormal level of total free floating DNA; and
j) concluding, whether a medical condition associated with said tissue, cell type or organ is present.

14. The method of claim 10, comprising the following additional step:
e) concluding, which kind of further diagnostic tests will have to be employed.

15. The method of claim 10, comprising the following additional step:
e) concluding, to which kind of specialist the patient might be referred to.

16. The method according to any of the preceding claims, characterised in that the total amount of free floating DNA is measured by intercalating fluorescent dyes or other dyes changing their fluorescence properties when binding to DNA, hybridisation to DNA specific probes including, but not limited to oligonucleotides or PNA oligomers, real time PCR assays or other real time amplification procedures, UV-Vis absorbance or in general amplification procedures with subsequent determination of the amount of product formed.

17. A method for diagnosing a disease or medical condition, comprising a method according to any of the preceding claims.

18. A kit for determining the total amount of free floating DNA in serum, comprising:
a surface to bind DNA floating in a sample volume of bodily fluid,
a means for detecting the amount of DNA bound to this solid surface,
reagents to chemically or enzymatically modify the DNA bound to the surface,
a container to host the surface and said reagents, and
a means to control and adjust the temperature in this chamber.

19. Use of the method according to the claims above for guiding a physicians' selection on employing further diagnostic tests.

20. Use of the method according to the claims above for guiding the decision of a general physicist to refer a patient to a specific kind of specialist.


SEQUENCE LISTING (8 sequences)

SEQ ID NO: 1 - DNA, 25 bp, Homo sapiens
tggtgatggaggaggtttagtaagt

SEQ ID NO: 2 - DNA, 27 bp, Homo sapiens
aaccaataaaacctactcctcccttaa

SEQ ID NO: 3 - DNA, 29 bp, Homo sapiens
accaccacccaacacacaataacaaacca

SEQ ID NO: 4 - DNA, 25 bp, Homo sapiens
ggtgattgtttattgttatggtttg

SEQ ID NO: 5 - DNA, 23 bp, Homo sapiens
cccctcaacctaaaaactacaac

SEQ ID NO: 6 - DNA, 25 bp, Homo sapiens
gttatggtttgtgattttgtgtggg

SEQ ID NO: 7 - DNA, 26 bp, Homo sapiens
aaactacaaccactcaaatcaaccca

SEQ ID NO: 8 - DNA, 25 bp, Homo sapiens
aaaattacgacgacgccacccgaaa